SEQUENCE LISTING (2 sequences)

SEQ ID NO: 1 - DNA, 49 bp, Artificial Sequence
  Description of Artificial Sequence  forward primer
tactatggtaccttaggaaacagctatgcccatatccaagatactcgtt

SEQ ID NO: 2 - DNA, 49 bp, Artificial Sequence
  Description of Artificial Sequence  reverse primer
attcgtactcaggatctgaaagatctaacagcctgactttacacaatcg

(19) United States
(12) Patent Application Publication
(10) Pub. No.: US 2003/0087381 A1
(43) Pub. Date: May 8, 2003

(54) Metabolically engineered organisms for enhanced production of oxaloacetate-derived biochemicals

(75) Inventors: Ravi R. Gokarn, Plymouth, MN (US); Mark A. Eiteman, Athens, GA (US); Elliot Altman, Athens, GA (US)

Correspondence Address: MUETING, RAASCH & GEBHARDT, P.A., P.O. BOX 581415, MINNEAPOLIS, MN, 55458, US

(73) Assignee: University of Georgia Research Foundation, Inc., Athens, GA (US)

(21) Appl. No.: 10/215,440

(22) Filed: Aug. 9, 2002

Related U.S. Application Data
(63) Continuation-in-part of application No. 09/417,557, filed on Oct. 13, 1999, now Pat. No. 6,455,284.
Application No. 09/417,557 is a continuation-in-part of application No. PCT/US99/08014, filed on Apr. 13, 1999.
(60) Provisional application No. 60/081,598, filed on Apr. 13, 1998.
(60) Provisional application No. 60/082,850, filed on Apr. 23, 1998.

(51) Int. Cl. C12P 21/02; C12N 1/21; C07H 21/04; C12N 9/10; C12N 15/74

(52) U.S. Cl. 435/69.1; 435/320.1; 435/193; 435/252.3; 435/252.33; 536/23.2

(57) ABSTRACT

Metabolic engineering is used to increase the carbon flow toward oxaloacetate to enhance production of bulk biochemicals, such as lysine and succinate, in industrial fermentations. Carbon flow is redirected by genetically engineering the cells to overexpress the enzyme pyruvate carboxylase.

This application is a continuation-in-part application of U.S. application Ser. No. 09/417,557, filed Oct. 13, 1999, which is a continuation-in-part of International Application PCT/US99/08014, with an international filing date of Apr. 13, 1999, which in turn claims the benefit of U.S. Provisional Application No. 60/081,598, filed Apr. 13, 1998, and U.S. Provisional Application No. 60/082,850, filed Apr. 23, 1998, each of which is incorporated herein by reference in its entirety.

### BACKGROUND OF THE INVENTION

Tremendous commercial potential exists for producing oxaloacetate-derived biochemicals via aerobic or anaerobic bacterial fermentation processes. Aerobic fermentation processes can be used to produce oxaloacetate-derived amino acids such as asparagine, aspartate, methionine, threonine, isoleucine, and lysine. Lysine, in particular, is of great commercial interest in the world market. Raw materials comprise a significant portion of lysine production cost, and hence process yield (product generated per substrate consumed) is an important measure of performance and economic viability. The stringent metabolic regulation of carbon flow (described below) can limit process yields. Carbon flux towards oxaloacetate (OAA) remains constant regardless of system perturbations (J. Vallino et al., Biotechnol. Bioeng., 41, 633-646 (1993)). In one reported fermentation, to maintain this rigid regulation of carbon flow at the low growth rates desirable for lysine production, the cells converted less carbon to oxaloacetate, thereby limiting the lysine yield (R. Kiss et al., Biotechnol. Bioeng., 39, 565-574 (1992)). Hence, a tremendous opportunity exists to improve the process by overcoming the metabolic regulation of carbon flow.

Anaerobic fermentation processes can be used to produce oxaloacetate-derived organic acids such as malate, fumarate, and succinate. Chemical processes using petroleum feedstock can also be used, and have historically been more efficient for production of these organic acids than bacterial fermentations. Succinic acid in particular, and its derivatives, have great potential for use as specialty chemicals. They can be advantageously employed in diverse applications in the food, pharmaceutical, and cosmetics industries, and can also serve as starting materials in the production of commodity chemicals such as 1,4-butanediol and tetrahydrofuran (L. Schilling, FEMS Microbiol. Rev., 16, 101-110 (1995)). Anaerobic rumen bacteria have been considered for use in producing succinic acid via bacterial fermentation processes, but these bacteria tend to lyse during the fermentation. More recently, the strict anaerobe Anaerobiospirillum succiniciproducens has been used, which is more robust and produces higher levels of succinate (R. Datta, U.S. Pat. No. 5,143,833 (1992); R. Datta et al., Eur. Pat. Appl. 405707 (1991)).

Commercial fermentation processes use crop-derived carbohydrates to produce bulk biochemicals. Glucose, one common carbohydrate substrate, is usually metabolized via the Embden-Meyerhof-Parnas (EMP) pathway, also known as the glycolytic pathway, to phosphoenolpyruvate (PEP) and then pyruvate. All organisms derive some energy from the glycolytic breakdown of glucose, regardless of whether they are grown aerobically or anaerobically. However, beyond these two intermediates, the pathways for carbon metabolism are different depending on whether the organism grows aerobically or anaerobically, and the fates of PEP and pyruvate depend on the particular organism involved as well as the conditions under which metabolism is taking place.

In aerobic metabolism, the carbon atoms of glucose are oxidized fully to carbon dioxide in a cyclic process known as the tricarboxylic acid (TCA) cycle or, sometimes, the citric acid cycle, or Krebs cycle. The TCA cycle begins when oxaloacetate combines with acetyl-CoA to form citrate. Complete oxidation of glucose during the TCA cycle ultimately liberates significantly more energy from a single molecule of glucose than is extracted during glycolysis alone. In addition to fueling the TCA cycle in aerobic fermentations, oxaloacetate also serves as an important precursor for the synthesis of the amino acids asparagine, aspartate, methionine, threonine, isoleucine and lysine. This aerobic pathway is shown in FIG. 1 for Escherichia coli, the most commonly studied microorganism. Anaerobic organisms, on the other hand, do not fully oxidize glucose. Instead, pyruvate and oxaloacetate are used as acceptor molecules in the reoxidation of reduced cofactors (NADH) generated in the EMP pathway. This leads to the generation and accumulation of reduced biochemicals such as acetate, lactate, ethanol, formate and succinate. This anaerobic pathway for E. coli is shown in FIG. 2.

Intermediates of the TCA cycle are also used in the biosynthesis of many important cellular compounds. For example, α-ketoglutarate is used to biosynthesize the amino acids glutamate, glutamine, arginine, and proline, and succinyl-CoA is used to biosynthesize porphyrins. Under anaerobic conditions, these important intermediates are still needed. As a result, succinyl-CoA, for example, is made under anaerobic conditions from oxaloacetate in a reverse reaction; i.e., the TCA cycle runs backwards from oxaloacetate to succinyl-CoA.

Oxaloacetate that is used for the biosynthesis of these compounds must be replenished if the TCA cycle is to continue unabated and metabolic functionality is to be maintained. Many organisms have thus developed what are known as “anaplerotic pathways” that regenerate intermediates for recruitment into the TCA cycle. Among the important reactions that accomplish this replenishing are those in which oxaloacetate is formed from either PEP or pyruvate. These pathways that resupply intermediates in the TCA cycle can be utilized during either aerobic or anaerobic metabolism.

PEP occupies a central position, or node, in carbohydrate metabolism. As the final intermediate in glycolysis, and hence the immediate precursor in the formation of pyruvate via the action of the enzyme pyruvate kinase, it can serve as a source of energy. Additionally, PEP can replenish intermediates in the TCA cycle via the anaplerotic action of the enzyme PEP carboxylase, which converts PEP directly into the TCA intermediate oxaloacetate. PEP is also often a cosubstrate for glucose uptake into the cell via the phosphotransferase system (PTS) and is used to biosynthesize aromatic amino acids. In many organisms, TCA cycle intermediates can be regenerated directly from pyruvate. For example, pyruvate carboxylase (PYC), which is found in some bacteria but not E. coli or Salmonella typhimurium, mediates the formation of oxaloacetate by the carboxylation of pyruvate utilizing carboxybiotin. As might be expected, the partitioning of PEP is rigidly regulated by cellular control mechanisms, causing a metabolic “bottleneck” which limits the amount and direction of carbon flowing through this juncture. The enzyme-mediated conversions that occur between PEP, pyruvate and oxaloacetate are shown in FIG. 3.

TCA cycle intermediates can also be regenerated in some plants and microorganisms from acetyl-CoA via what is known as the “glyoxylate shunt,” “glyoxylate bypass” or glyoxylate cycle (FIG. 4). This pathway enables organisms growing on 2-carbon substrates to replenish their oxaloacetate. Examples of 2-carbon substrates include acetate and other fatty acids as well as long-chain n-alkanes. These substrates do not provide a 3-carbon intermediate such as PEP which can be carboxylated to form oxaloacetate. In the glyoxylate shunt, isocitrate from the TCA cycle is cleaved into glyoxylate and succinate by the enzyme isocitrate lyase. The released glyoxylate combines with acetyl-CoA to form malate through the action of the enzyme malate synthase. Both succinate and malate generate oxaloacetate through the TCA cycle. Expression of the genes encoding the glyoxylate bypass enzymes is tightly controlled, and normally these genes are repressed when 3-carbon compounds are available. In E. coli, for example, the genes encoding the glyoxylate bypass enzymes are located on the aceBAK operon and are controlled by several transcriptional regulators: iclR (A. Sunnarborg et al., J. Bacteriol., 172, 2642-2649 (1990)), fadR (S. Maloy et al., J. Bacteriol., 148, 83-90 (1981)),fruR (A. Chin et al., J. Bacteriol., 171, 2424-2434 (1989)), and arcAB (S. Iuchi et al., J. Bacteriol., 171, 868-873 (1989); S. Iuchi et al., Proc. Natl. Acad. Sci. USA, 85, 1888-1892 (1988)). The glyoxylate bypass enzymes are not expressed when E. coli is grown on glucose, glycerol, or pyruvate as a carbon source. The glyoxylate shunt is induced by fatty acids such as acetate (Kornberg, Biochem. J., 99, 1-11 (1966)).

Various metabolic engineering strategies have been pursued, with little success, in an effort to overcome the network rigidity that surrounds carbon metabolism. For example, overexpression of the native enzyme PEP carboxylase in E. coli was shown to increase the carbon flux towards oxaloacetate (C. Millard et al., Appl. Environ. Microbiol., 62, 1808-1810 (1996); W. Farmer et al, Appl. Env. Microbiol., 63, 3205-3210 (1997)); however, such genetic manipulations also cause a decrease in glucose uptake (P. Chao et al., Appl. Env. Microbiol., 59, 4261-4265 (1993)), since PEP is a required cosubstrate for glucose transport via the phosphotransferase system. An attempt to improve lysine biosynthesis in Corynebacterium glutamicum by overexpressing PEP carboxylase was likewise not successful (J. Cremer et al., Appl. Env. Microbiol., 57, 1746-1752 (1991)). In another approach to divert carbon flow toward oxaloacetate, the glyoxylate shunt in E. coli was derepressed by knocking out one of the transcriptional regulators, fadR. Only a slight increase in biochemicals derived from oxaloacetate was observed (W. Farmer et al., Appl. Environ. Microbiol., 63, 3205-3210 (1997)). In a different approach, malic enzyme from Ascaris suum was overproduced in mutant E. coli which were deficient for the enzymes that convert pyruvate to lactate, acetyl-CoA, and formate. This caused pyruvate to be converted to malate which increased succinate production (see FIG. 2). However, this approach is problematic, since the mutant strain in question cannot grow under the strict anaerobic conditions which are required for the optimal fermentation of glucose to organic acids (L. Stols et al., Appl. Biochem. Biotechnol., 63-65, 153-158 (1997)).

A metabolic engineering approach that successfully overcomes the network rigidity that characterizes carbon metabolism and diverts more carbon toward oxaloacetate, thereby increasing the yields of oxaloacetate-derived biochemicals per amount of added glucose, would represent a significant and long awaited advance in the field.

### SUMMARY OF THE INVENTION

The present invention employs a unique metabolic engineering approach which overcomes a metabolic limitation that cells use to regulate the synthesis of the biochemical oxaloacetate. The invention utilizes metabolic engineering to divert more carbon from pyruvate to oxaloacetate by making use of the enzyme pyruvate carboxylase. This feat can be accomplished by introducing a native (i.e., endogenous) and/or foreign (i.e., heterologous) nucleic acid fragment which encodes a pyruvate carboxylase into a host cell, such that a functional pyruvate carboxylase is overproduced in the cell. Alternatively, the DNA of a cell that endogenously expresses a pyruvate carboxylase can be mutated to alter transcription of the native pyruvate carboxylase gene so as to cause overproduction of the native enzyme. For example, a mutated chromosome can be obtained by employing either chemical or transposon mutagenesis and then screening for mutants with enhanced pyruvate carboxylase activity using methods that are well-known in the art. Overexpression of pyruvate carboxylase causes the flow of carbon to be preferentially diverted toward oxaloacetate and thus increases production of biochemicals which are biosynthesized from oxaloacetate as a metabolic precursor.

Accordingly, the present invention provides a metabolically engineered cell that overexpresses pyruvate carboxylase. Overexpression of pyruvate carboxylase is preferably effected by transforming the cell with a DNA fragment encoding a pyruvate carboxylase that is derived from an organism that endogenously expresses pyruvate carboxylase, such as Rhizobium etli, Corynebacterium glutamicum, Methanobacterium thermoautotrophicum, or Pseudomonas fluorescens. Pyruvate carboxylase can be expressed within the engineered cell from an expression vector, or alternatively from a DNA fragment that has been chromosomally integrated into the cell's genome. Optionally, the metabolically engineered cell of the invention overexpresses PEP carboxylase in addition to pyruvate carboxylase. Also optionally, the metabolically engineered cell does not express a detectable level of PEP carboxykinase. In a particularly preferred embodiment of the invention, the metabolically engineered cell is a C. glutamicum, E. coli, S. typhimurium, Brevibacterium flavum, or Brevibacterium lactofermentum cell that expresses a heterologous pyruvate carboxylase.

The invention also includes a method for making a metabolically engineered cell that involves transforming a cell with a nucleic acid fragment that contains a nucleotide sequence encoding an enzyme having pyruvate carboxylase activity, to yield a metabolically engineered cell that overexpresses pyruvate carboxylase. The method optionally includes co-transforming the cell with a nucleic acid fragment that contains a nucleotide sequence encoding an enzyme having PEP carboxylase activity so that the metabolically engineered cells also overexpress PEP carboxylase.

Also included in the invention is a method for making an oxaloacetate-derived biochemical that includes providing a cell that produces the biochemical; transforming the cell with a nucleic acid fragment containing a nucleotide sequence encoding an enzyme having pyruvate carboxylase activity; expressing the enzyme in the cell to cause increased production of the biochemical; and isolating the biochemical from the cell. Preferred biochemicals having oxaloacetate as a metabolic precursor include, but are not limited to, amino acids such as lysine, asparagine, aspartate, methionine, threonine, and isoleucine; organic acids such as succinate, malate and fumarate; pyrimidine nucleotides; and porphyrins.

The invention further includes a nucleic acid fragment isolated from P. fluorescens which contains a nucleotide sequence encoding a pyruvate carboxylase enzyme, preferably the α4β4 pyruvate carboxylase enzyme produced by P. fluorescens.

### BRIEF DESCRIPTION OF THE DRAWINGS

FIG. 1. Aerobic pathway in E. coli depicting glycolysis, the TCA cycle, and biosynthesis of oxaloacetate-derived biochemicals; dashed lines signify that multiple steps are required to biosynthesize the compound while solid lines signify a one-step conversion; the participation of PEP in glucose uptake is shown by a light line; the pathway as shown is not stoichiometric, nor does it include cofactors.

FIG. 2. Anaerobic pathway in E. coli depicting glycolysis and biosynthesis of selected oxaloacetate-derived biochemicals; the participation of PEP in glucose uptake is shown by the dashed line; the pathway as shown is not stoichiometric, nor does it include all cofactors.

FIG. 3. Biosynthetic pathways that directly regulate the intracellular levels of oxaloacetate; not all organisms contain all of these enzymes; E. coli, for example, does not contain pyruvate carboxylase.

FIG. 4. The TCA cycle, showing entry into the cycle of 3-carbon intermediates and also including the glyoxylate shunt for 2-carbon intermediates (darker arrows).

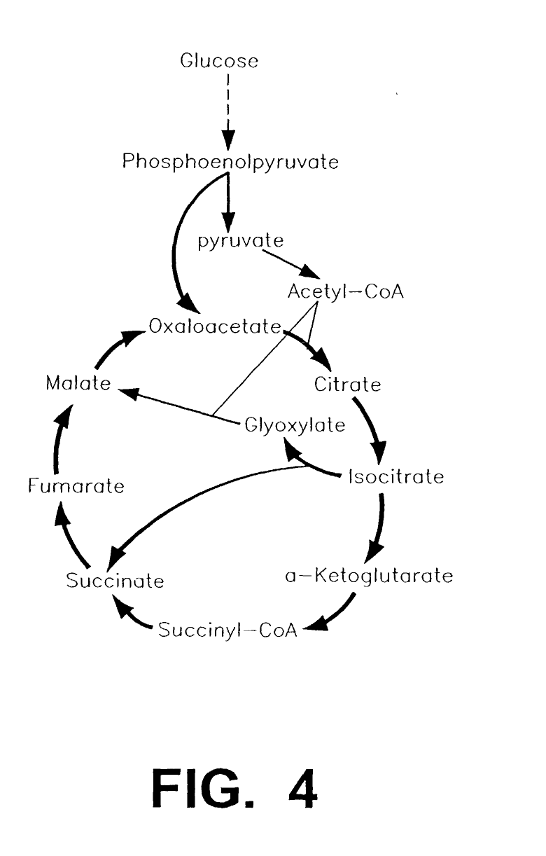

FIG. 5. Kinetic analysis of pyruvate carboxylase activities for MG 1655 pUC18 (◯) and MG1655 pUC18-pyc (●) with respect to pyruvate.

FIG. 6. Effects of increasing aspartate concentrations on the activity of pyruvate carboxylase.

FIG. 7. Kinetic analysis of pyruvate carboxylase with respect to ATP and ADP; pyruvate carboxylase activity was determined in the absence of ADP (●) and in the presence of 1.5 mM ADP (◯).

FIG. 8. Growth of a ppc null E. coli strain which contains either pUC18 or the pUC18-pyc construct on minimal media that utilizes glucose as a sole carbon source.

FIG. 9. Effect of nicotinamide nucleotides on pyruvate carboxylase activity: NADH (◯), NAD+(□), NADPH (Δ) and NADP+(□).

FIG. 10. Growth pattern and selected fermentation products of wild-type strain (MG1655) under strict anaerobic conditions in a glucose-limited (10 g/L) medium; concentrations of glucose (●), succinate (▪), lactate (◯), formate (□) and dry cell mass (Δ) were measured.

FIG. 11. Growth pattern and selected fermentation products of wild-type strain with pUC18 cloning/expression vector (MG1655/pUC18) under strict anaerobic conditions in a glucose-limited (10 g/L) medium; concentrations of glucose (●), succinate (▪), lactate (◯), formate (□) and dry cell mass (Δ) were measured.

FIG. 12. Growth pattern and selected fermentation products of wild-type strain with pyc gene (MG1655/pUC18-pyc) under strict anaerobic conditions in a glucose-limited (10 g/L) medium; concentrations of glucose (●), succinate (▪), lactate (◯), formate (□) and dry cell mass (Δ) were measured.

FIG. 13. Growth pattern and threonine production in the threonine producing strain βIM-4 (ATCC 21277) containing either pTrc99A or pTrc99A-pyc under strict aerobic conditions in a glucose-limited (30 g/L) medium; optical density in the pTrc99A containing strain (◯), optical density in the pTrc99A-pyc containing strain (□),threonine concentrations in the pTrc99A containing strain (●), and threonine concentrations in the pTrc99A-pyc containing strain (▪) were measured.

FIG. 14. Concentrations of glucose (◯, ●), succinate (□, ▪) and pyruvate (Δ,▴) from the exclusively anaerobic fermentations of E. coli NZN111 open symbols) and AFP111 (solid symbols) on glucose-rich media. The

FIG. 15. Concentrations of glucose (◯, ●), succinate (□, ▪) and pyruvate (Δ,▴) from the exclusively anaerobic fermentations of E. coli NZN111-pyc (open symbols) and AFP111-pyc (solid symbols) on glucose-rich media. The strains were not induced with IPTG at the onset of these fermentations.

FIG. 16. Concentrations of glucose (◯, ●), succinate (□, ▪), pyruvate (Δ,▴) and fumarate (♦) from the exclusively anaerobic fermentations of E. coli NZN111-pyc (open symbols) and AFP111-pyc (solid symbols) on glucose-rich media. The strains were induced with 1.0 mM IPTG at the onset of the fermentations.

FIG. 17. Aerobic fermentation of AFP111 at a medium value of k_{L}a (52 h⁻¹). (A) dry cell weight (DCW) (Δ), dissolved oxygen concentration (DO) (◯) and respiratory quotient (RQ) (▪). (B) The specific activities of the key enzymes: glucokinase (●), PEP carboxylase (□), pyruvate dehydrogenase (▾), isocitrate lyase (∇) and fumarate reductase (⋄). Milestone (1) is shown.

FIG. 18. Aerobic fermentation of AFP111 at a high value of k_{L}a (69 h⁻¹). (A) dry cell weight (DCW) (Δ), dissolved oxygen concentration (DO) (◯) and respiratory quotient (RQ) (▪). (B) The specific activities of the key enzymes: glucokinase (●), PEP carboxylase (□), pyruvate dehydrogenase (▾), isocitrate lyase (∇) and fumarate reductase (⋄). Milestones (2) and (3) are shown.

FIG. 19. Aerobic fermentation of AFP111/pTrc99A-pyc at a medium value of k_{L}a (52 h⁻¹). (A) dry cell weight (DCW) (Δ), dissolved oxygen concentration (DO) (◯) and Respiratory Quotient (RQ) (▪). (B) The specific activities of the key enzymes: glucokinase (●), PEP carboxylase (□), pyruvate carboxylase (▴), pyruvate dehydrogenase (▾), isocitrate lyase (∇) and fumarate reductase (⋄). Milestones (4) and (5) are shown.

FIG. 20. Aerobic fermentation of AFP111/pTrc99A-pyc at a high value of k_{L}a (69 h⁻¹). (A) Dry Cell Weight (DCW) (Δ), Dissolved Oxygen concentration (DO) (◯) and Respiratory Quotient (RQ) (▪). (B) The specific activities of the key enzymes: glucokinase (●), PEP carboxylase (□), pyruvate carboxylase (▴), pyruvate dehydrogenase (▾), isocitrate lyase (∇) and fumarate reductase (⋄). Milestone (6) is shown.

FIG. 21. Fed-batch dual-phase fermentation of AFP111/pTrc99A-pyc at a medium value of k_{L}a (52 h⁻¹) using milestone #4 as the time of transition. Glucose (◯), succinate (●), acetate (□), ethanol (▴) concentrations are shown.

FIG. 22. Glucose and selected product concentrations of S. typhimurium LT2 grown in a glucose rich medium: glucose (●) succinate (□); lactate (▴); formate, (♦).

FIG. 23. Glucose and selected product concentrations of S. typhimurium LT2-pyc grown in a glucose rich medium: glucose, ●; succinate, □; lactate, ▴; formate, (♦).

### DETAILED DESCRIPTION OF ILLUSTRATIVE EMBODIMENTS

Metabolic engineering involves genetically overexpressing particular enzymes at critical points in a metabolic pathway, and/or blocking the synthesis of other enzymes, to overcome or circumvent metabolic “bottlenecks.” The goal of metabolic engineering is to optimize the rate and conversion of a substrate into a desired product. The present invention employs a unique metabolic engineering approach which overcomes a metabolic limitation that cells use to regulate the synthesis of the biochemical oxaloacetate. Specifically, cells of the present invention are genetically engineered to overexpress a functional pyruvate carboxylase, resulting in increased levels of oxaloacetate.

Genetically engineered cells are referred to herein as “metabolically engineered” cells when the genetic engineering is directed to disruption or alteration of a metabolic pathway so as to cause a change in the metabolism of carbon. An enzyme is “overexpressed” in a metabolically engineered cell when the enzyme is expressed in the metabolically engineered cell at a level higher than the level at which it is expressed in a comparable wild-type cell. In cells that do not endogenously express a particular enzyme, any level of expression of that enzyme in the cell is deemed an “overexpression” of that enzyme for purposes of the present invention.

Many organisms can synthesize oxaloacetate from either PEP via the enzyme PEP carboxylase, or from pyruvate via the biotin-dependent enzyme pyruvate carboxylase. Representatives of this class of organisms include C. glutamicum, R. etli, P. fluorescens, Pseudomonas citronellolis, Azotobacter vinelandii, Aspergillus nidulans, and rat liver cells. Other organisms cannot synthesize oxaloacetate directly from pyruvate because they lack the enzyme pyruvate carboxylase. E. coli, S. typhimurium, Fibrobacter succinogenes, and Ruminococcus flavefaciens are representatives of this class of organisms. In either case, the metabolic engineering approach of the present invention can be used to redirect carbon to oxaloacetate and, as a result, enhance the production of biochemicals which use oxaloacetate as a metabolic precursor.

The cell that is metabolically engineered according to the invention is not limited in any way to any particular type or class of cell. It can be a eukaryotic cell or a prokaryotic cell; it can include, but is not limited to, a cell of a human, animal, plant, insect, yeast, protozoan, bacterium, or archaebacterium. Preferably, the cell is a microbial cell, more preferably, a bacterial cell, particularly a gram-negative bacterial cell such as those from the genus Escherichia, Salmonella and Serratia. Advantageously, the bacterial cell can be an E. coli, C. glutamicum, S. typhimurium, B. flavum or B. lactofermentum cell; these strains are currently being employed industrially to make amino acids which can be derived from oxaloacetate using bacterial fermentation processes. Mutant E. coli strains are currently being considered for commercial synthesis of succinate via anaerobic fermentation (L. Stols et al., Appl. Environ. Microbiol., 63, 2695-2701 (1997); L. Stols et al., Appl. Biochem. Biotech., 63, 153-158 (1997)), although A. succiniciproducens has been considered in the past. Rhizopus fungi are now being considered to produce fumarate via aerobic fermentations (N. Cao, Appl. Biochem. Biotechnol., 63, 387-394 (1997); J. Du et al., Appl. Biochem. Biotech., 63, 541-556 (1997)). Bacteria that lack endogenous pyruvate carboxylase, such as E. coli, S. typhimurium, Fibrobacter succinogenes, and R. flaveflaciens, can be used in the metabolic engineering strategy described by the invention.

Optionally, the metabolically engineered cell has been engineered to disrupt, block, attenuate or inactivate one or more metabolic pathways that draw carbon away from oxaloacetate. For example, alanine and valine can typically be biosynthesized directly from pyruvate, and by inactivating the enzymes involved in the synthesis of either or both of these amino acids, oxaloacetate production can be increased. Thus, the metabolically engineered cell of the invention can be an alanine and/or a valine auxotroph, more preferably a C. glutamicum alanine and/or a valine auxotroph. Likewise, the metabolically engineered cell can be engineered to reduce or eliminate the production of PEP carboxykinase, which catalyzes the formation of PEP from oxaloacetate (the reverse of the reaction catalyzed by PEP carboxylase). Preventing or reducing the expression of a functional PEP carboxykinase will result in more carbon shunted to oxaloacetate and hence the amino acids and organic acids biosynthesized therefrom.

Another alternative involves interfering with the metabolic pathway used to produce acetate from acetyl CoA. Disrupting this pathway should result in higher levels of acetyl CoA, which may then indirectly result in increased amounts of oxaloacetate. Moreover, where the pyruvate carboxylase enzyme that is expressed in the metabolically engineered cell is one that is activated by acetyl CoA (see below), higher levels of acetyl CoA in these mutants lead to increased activity of the enzyme, causing additional carbon to flow from pyruvate to oxaloacetate. Thus, acetate-mutants are preferred metabolically engineered cells.

The pyruvate carboxylase expressed by the metabolically engineered cell can be either endogenous or heterologous. A “heterologous” enzyme is one that is encoded by a nucleotide sequence that is not normally present in the cell. For example, a bacterial cell that has been transformed with and expresses a gene from a different species or genus that encodes a pyruvate carboxylase contains a heterologous pyruvate carboxylase. The heterologous nucleic acid fragment may or may not be integrated into the host genome. The ter “pyruvate carboxylase” means a molecule that has pyruvate carboxylase activity; i.e., that is able to catalyze carboxylation of pyruvate to yield oxaloacetate. The term“pyruvate carboxylase” thus includes naturally occurring pyruvate carboxylase enzymes, along with fragments, derivatives, or other chemical, enzymatic or structural modifications thereof, including enzymes encoded by insertion, deletion or site mutants of naturally occurring pyruvate carboxylase genes, as long as pyruvate carboxylase activity is retained. Pyruvate carboxylase enzymes and, in some cases, genes that have been characterized include human pyruvate carboxylase (GenBank K02282; S. Freytag et al., J. Biol. Chem., 259, 12831-12837 (1984)); pyruvate carboxylase from Saccharomyces cerevisiae (GenBank X59890, J03889, and M16595; R. Stucka et al., Mol. Gen. Genet., 229, 307-315 (1991); F. Lim et al., J. Biol. Chem., 263, 11493-11497 (1988); D. Myers et al., Biochemistry, 22, 5090-5096 (1983)); pyruvate carboxylase from Schizosaccharomyces pombe (Gen bank D78170); pyruvate carboxylase from R. etli (GenBank U51439; M. Dunn et al., J. Bacteriol., 178, 5960-5070 (1996)); pyruvate carboxylase from Rattus norvegicus (GenBank U81515; S. Jitrapakdee et al., J. Biol. Chem., 272, 20522-20530 (1997)); pyruvate carboxylase from Bacillus stearothermophilis (GenBank D83706; H. Kondo, Gene, 191, 47-50 (1997); S. Libor, Biochemistry, 18, 3647-3653 (1979)); pyruvate carboxylase from P. fluorescens (R. Silvia et al., J. Gen. Microbiol., 93, 75-81 (1976); pyruvate carboxylase from M. thermoautotrophicum (1998)); and pyruvate carboxylase from C. glutamicum (GenBank Y09548).

Preferably, the pyruvate carboxylase expressed by the metabolically engineered cells is derived from either R. etli or P. fluorescens. The pyruvate carboxylase in R. etli is encoded by the pyc gene (M. Dunn et al., J. Bacteriol., 178, 5960-5970 (1996)). The R. etli enzyme is classified as an α4 pyruvate carboxylase, which is inhibited by aspartate and requires acetyl CoA for activation. Members of this class of pyruvate carboxylases might not seem particularly well-suited for use in the present invention, since redirecting carbon flow from pyruvate to oxaloacetate would be expected to cause reduced production of acetyl CoA, and increased production of aspartate, both of which will decrease pyruvate carboxylase activity. However, expression of R. etli pyruvate carboxylase in a bacterial host is shown herein to be effective to increase production of oxaloacetate and its downstream metabolites (see Examples I and II). Moreover, this can be accomplished without adversely affecting glucose uptake by the host (see Example III) which has been the stumbling block in previous efforts to divert carbon to oxaloacetate by overexpressing PEP carboxylase (P. Chao et al., Appl. Env. Microbiol., 59, 4261-4265 (1993)).

In a particularly preferred embodiment, the metabolically engineered cell expresses an α4β4 pyruvate carboxylase. Members of this class of pyruvate carboxylases do not require acetyl CoA for activation, nor are they inhibited by aspartate, rendering them particularly well-suited for use in the present invention. P. fluorescens is one organism known to expresses an α4β4 pyruvate carboxylase. The metabolically engineered cell of the invention therefore is preferably one that has been transformed with a nucleic acid fragment isolated from P. fluorescens which contains a nucleotide sequence encoding a pyruvate carboxylase expressed therein, more preferably the pyruvate carboxylase isolated and described in S. Milrad de Forchetti et al., J. Gen. Microbiol., 93, 75-81 (1976), which is incorporated herein by reference, in its entirety.

Accordingly, the invention also includes a nucleic acid fragment isolated from P. fluorescens which includes a nucleotide sequence encoding a pyruvate carboxylase, more preferably a nucleotide sequence that encodes the pyruvate carboxylase isolated and described in S. Milrad de Forchetti et al., J. Gen. Microbiol., 93, 75-81 (1976).

The metabolically engineered cell of the invention is made by transforming a host cell with a nucleic acid fragment comprising a nucleotide sequence encoding an enzyme having pyruvate carboxylase activity. Methods of transformation for bacteria, plant, and animal cells are well known in the art. Common bacterial transformation methods include electroporation and chemical modification. Transformation yields a metabolically engineered cell that overexpresses pyruvate carboxylase. The preferred cells and pyruvate carboxylase enzymes are as described above in connection with the metabolically engineered cell of the invention. Optionally, the cells are further transformed with a nucleic acid fragment comprising a nucleotide sequence encoding an enzyme having PEP carboxylase activity to yield a metabolically engineered cell that also overexpresses pyruvate carboxylase, also as described above. The invention is to be broadly understood as including methods of making the various embodiments of the metabolically engineered cells of the invention described herein.

Preferably, the nucleic acid fragment is introduced into the cell using a vector, although “naked DNA” can also be used. The nucleic acid fragment can be circular or linear, single-stranded or double stranded, and can be DNA, RNA, or any modification or combination thereof. The vector can be a plasmid, a viral vector or a cosmid. Selection of a vector or plasmid backbone depends upon a variety of desired characteristics in the resulting construct, such as a selection marker, plasmid reproduction rate, and the like. Suitable plasmids for expression in E. coli, for example, include pUC(X), pKK223-3, pKK233-2, pTrc99A, and pET-(X) wherein (X) denotes a vector family in which numerous constructs are available. pUC(X) vectors can be obtained from Pharmacia Biotech (Piscataway, N.H.) or Sigma Chemical Co. (St. Louis, Mo.). pKK223-3, pKK233-2 and pTrc99A can be obtained from Pharmacia Biotech. pET-(X) vectors can be obtained from Promega (Madison, Wis.) Stratagene (La Jolla, Calif.) and Novagen (Madison, Wis.). To facilitate replication inside a host cell, the vector preferably includes an origin of replication (known as an “ori ”) or replicon. For example, ColE1 and P15A replicons are commonly used in plasmids that are to be propagated in E. coli.

The nucleic acid fragment used to transform the cell according to the invention can optionally include a promoter sequence operably linked to the nucleotide sequence encoding the enzyme to be expressed in the host cell. A promoter is a DNA fragment which causes transcription of genetic material. Transcription is the formation of an RNA chain in accordance with the genetic information contained in the DNA. The invention is not limited by the use of any particular promoter, and a wide variety are known. Promoters act as regulatory signals that bind RNA polymerase in a cell to initiate transcription of a downstream (3′ direction) coding sequence. A promoter is “operably linked” to a nucleic acid sequence if it is does, or can be used to, control or regulate transcription of that nucleic acid sequence. The promoter used in the invention can be a constitutive or an inducible tac promoter. It can be, but need not be, heterologous with respect to the host cell. Preferred promoters for bacterial transformation include lac, lacUV5, tac, trc, T7, SP6 and ara.

The nucleic acid fragment used to transform the host cell can, optionally, include a Shine Dalgarno site (e.g., a ribosome binding site) and a start site (e.g., the codon ATG) to initiate translation of the transcribed message to produce the enzyme. It can, also optionally, include a termination sequence to end translation. A termination sequence is typically a codon for which there exists no corresponding aminoacetyl-tRNA, thus ending polypeptide synthesis. The nucleic acid fragment used to transform the host cell can optionally further include a transcription termination sequence. The rrnB terminators, which is a stretch of DNA that contains two terminators, T1 and T2, is the most commonly used terminator that is incorporated into bacterial expression systems (J. Brosius et al., J. Mol. Biol., 148, 107-127 (1981)).

The nucleic acid fragment used to transform the host cell optionally includes one or more marker sequences, which typically encode a gene product, usually an enzyme, that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol and tetracycline.

Pyruvate carboxylase can be expressed in the host cell from an expression vector containing a nucleic acid fragment comprising the nucleotide sequence encoding the pyruvate carboxylase. Alternatively, the nucleic acid fragment comprising the nucleotide sequence encoding pyruvate carboxylase can be integrated into the host's chromosome. Nucleic acid sequences, whether heterologous or endogenous with respect to the host cell, can be introduced into a bacterial chromosome using, for example, homologous recombination. First, the gene of interest and a gene encoding a drug resistance marker are inserted into a plasmid that contains piece of DNA that is homologous to the region of the chromosome within which the gene of interest is to be inserted. Next this recombinagenic DNA is introduced into the bacteria, and clones are selected in which the DNA fragment containing the gene of interest and drug resistant marker has recombined into the chromosome at the desired location. The gene and drug resistant marker can be introduced into the bacteria via transformation either as a linearized piece of DNA that has been prepared from any cloning vector, or as part of a specialized recombinant suicide vector that cannot replicate in the bacterial host. In the case of linearized DNA, a recD⁻ host can be used to increase the frequency at which the desired recombinants are obtained. Clones are then verified using PCR and primers that amplify DNA across the region of insertion. PCR products from non-recombinant clones will be smaller in size and only contain the region of the chromosome where the insertion event was to take place, while PCR products from the recombinant clones will be larger in size and contain the region of the chromosome plus the inserted gene and drug resistance.

In a preferred embodiment, the host cell, preferably E. coli, C. glutamicum, S. typhimurium, B. flavum or B. lactofermentum, is transformed with a nucleic acid fragment comprising a pyruvate carboxylase gene, preferably a gene that is isolated from R. etli or P. fluorescens, more preferably the pyc gene from R. etli, such that the gene is transcribed and expressed in the host cell to cause increased production of oxaloacetate and, consequently, increased production of the downstream metabolite of interest, relative to a comparable wild-type cell.

The metabolically engineered cell of the invention overexpresses pyruvate carboxylase. Stated in another way, the metabolically engineered cell expresses pyruvate carboxylase at a level higher than the level of pyruvate carboxylase expressed in a comparable wild-type cell. This comparison can be made in any number of ways by one of skill in the art and is done under comparable growth conditions. For example, pyruvate carboxylase activity can be quantified and compared using the method of Payne and Morris (J. Gen. Microbiol., 59, 97-101 (1969)). The metabolically engineered cell that overexpresses pyruvate carboxylase will yield a greater activity than a wild-type cell in this assay. In addition, or alternatively, the amount of pyruvate carboxylase can be quantified and compared by preparing protein extracts from the cells, subjecting them to SDS-PAGE, transferring them to a Western blot, then detecting the biotinylated pyruvate carboxylase protein using detection kits which are commercial available from, for example, Pierce Chemical Company (Rockford, Ill.), Sigma Chemical Company (St. Louis, Mo.) or Boehringer Mannheim (Indianapolis, Ind.) for visualizing biotinylated proteins on Western blots. In some suitable host cells, pyruvate carboxylase expression in the non-engineered, wild-type cell may be below detectable levels.

Optionally, the metabolically engineered cell of the invention also overexpresses PEP carboxylase. In other words, the metabolically engineered cell optionally expresses PEP carboxylase at a level higher than the level of PEP carboxylase expressed in a comparable wild-type cell. Again, this comparison can be made in any number of ways by one of skill in the art and is done under comparable growth conditions. For example, PEP carboxylase activity can be assayed, quantified and compared. In one assay, PEP carboxylase activity is measured in the absence of ATP using PEP instead of pyruvate as the substrate, by monitoring the appearance of CoA-dependent thionitrobenzoate formation at 412 nm (see Example III). The metabolically engineered cell that overexpresses PEP carboxylase will yield a greater PEP carboxylase activity than a wild-type cell. In addition, or alternatively, the amount of PEP carboxylase can be quantified and compared by preparing protein extracts from the cells, subjecting them to SDS-PAGE, transferring them to a Western blot, then detecting the PEP carboxylase protein using PEP antibodies in conjunction with detection kits available from Pierce Chemical Company (Rockford Ill.), Sigma Chemical Company (St. Louis, Mo.) or Boehringer Mannheim (Indianapolis, Ind.) for visualizing antigen-antibody complexes on Western blots. In a preferred embodiment, the metabolically engineered cell expresses PEP carboxylase derived from a cyanobacterium, more preferably Anacystis nidulans.

The invention further includes a method for producing an oxaloacetate-derived biochemical by enhancing or augmenting production of the biochemical in a cell that is, prior to transformation as described herein, capable of biosynthesizing the biochemical. The cell is transformed with a nucleic acid fragment comprising a nucleotide sequence encoding an enzyme having pyruvate carboxylase activity, the enzyme is expressed in the cell so as to cause increased production of the biochemical relative to a comparable, wild-type cell, and the biochemical is isolated from the cell according to known methods. The biochemicals can be isolated from the metabolically engineered cells using protocols, methods and techniques that are well-known in the art. For example, succinic acid can be isolated by electrodialysis (D. Glassner et al., U.S. Pat. No. 5,143,834 (1992)) or by precipitation as calcium succinate (R. Datta, U.S. Pat. No., 5,143,833 (1992)); malic acid can be isolated by electrodialysis (R. Sieipenbusch, U.S. Pat. No. 4,874,700 (1989)); lysine can be isolated by adsorption/reverse osmosis (T. Kaneko et al., U.S. Pat. No. 4,601,829 (1986)). The preferred host cells, oxaloacetate-derived biochemicals, and pyruvate carboxylase enzymes are as described herein.

The metabolically engineered cells can be cultured aerobically or anaerobically, or in a multiple phase fermentation that makes use of periods of anaerobic and aerobic fermentation. For example, the cells can be grown aerobically for biomass generation then subjected to anaerobic conditions to produce the desired biochemical(s) (a “dual-phase” fermentation). Dual-phase fermentations have the advantage of uncoupling growth and product formation, and thus unique operational conditions may be applied to each phase. Additionally, enzymes that carry out the biotransformations in the second non-growth production phase are largely expressed during the aerobic growth phase and remain active throughout the production phase. Dual-phase fermentations are therefore not limited by the expression of only a select set of anaerobically-induced enzymes, as in the case for example of a conventional exclusively anaerobic fermentation for succinate production by E. coli.

The biochemicals that are produced or overproduced in, and isolated from, the metabolically engineered cells according to the method of the invention are those that are or can be metabolically derived from oxaloacetate (i.e., with respect to which oxaloacetate is a metabolic precursor). These oxaloacetate-derived biochemicals include, but are not limited to, amino acids such as lysine, asparagine, aspartate, methionine, threonine, arginine, glutamate, glutamine, proline and isoleucine; organic acids such as succinate, malate, citrate, isocitrate, α-ketoglutarate, succinyl-CoA and fumarate; pyrimidine nucleotides; and porphyrins such as cytochromes, hemoglobins, chlorophylls, and the like. It is to be understood that the terms used herein to describe acids (for example, the terms succinate, aspartate, glutamate, malate, fumarate, and the like) are not meant to denote any particular ionization state of the acid, and are meant to include both protonated and unprotonated forms of the compound. For example, the terms aspartate and aspartic acid refer to the same compound and are used interchangeably, as well as succinate and succinic acid, malate and malic acid, fumarate and fumaric acid, and so on. As is well-known in the art, the protonation state of the acid depends on the pKₐ of the acidic group and the pH of the medium. At neutral pH, the acids described herein are typically unprotonated. Additionally, an oxaloacetate-derived biochemical includes a salt of the biochemical. The term succinate, for example, includes succinate salts such as potassium succinate, diammonium succinate, and sodium succinate.

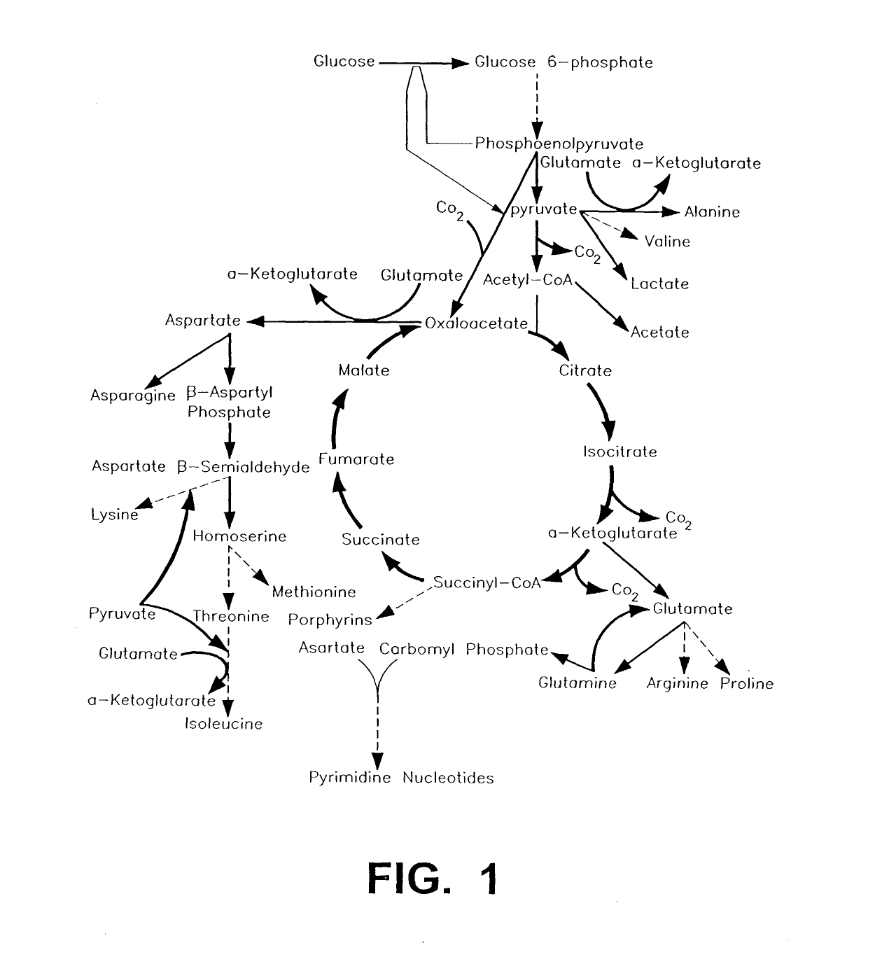

In a particularly preferred method, lysine and succinate are produced in and obtained from a metabolically engineered bacterial cell that expresses pyruvate carboxylase, preferably pyruvate carboxylase derived from either R. etli or P. fluorescens. The method of the invention is to be broadly understood to include the production and isolation of any or all oxaloacetate-derived biochemicals recovered or recoverable from the metabolically engineered cells of the invention, regardless of whether the biochemicals are actually synthesized from oxaloacetate in accordance with the metabolic pathways shown in FIGS. 1-3 or any other presently known metabolic pathways.

Advantages of the invention are illustrated by the following examples. However, the particular materials and amounts thereof recited in these examples, as well as other conditions and details, are to be interpreted to apply broadly in the art and should not be construed to unduly restrict or limit the invention in any way.

### EXAMPLE I

### Expression of the R. etli Pyruvate Carboxylase Enzyme Enables E. coli to Convert Pyruvate to Oxaloacetate

Materials and Methods

Bacterial strains, plasmids and growth conditions. The bacterial strains and plasmids used in this study are listed in Table 1. E. coli strains were grown in LB Miller broth (rich) or M9 minimal media (J. Miller, Experiments in Molecular Genetics, Cold Spring Harbor Laboratory, Cold Spring Harbor, N.Y. (1972)). Strains carrying a plasmid were supplemented with the appropriate antibiotic to detect the marker gene; ampicillin was used at 100 μg/ml in rich media and 50 μ/ml in minimal media while chloramphenicol was used at 20 μg/ml in rich media and 10 μg/ml in minimal media. When isopropyl β-D-thiogalactopyranoside (IPTG) was used to induce the pUC18-pyc construct, it was added at a final concentration of 1 mM.

TABLE 1
Strains and Plasmids

| Strains | Genotype | Reference or source |
|---|---|---|
| MC1061 | araD139 Δ(araABOIC- | M. Casadaban et al., J. Mol. Biol, 138, 179- |
| | leu)7679 Δ(lac)74 galU galK | 207 (1980) |
| | rpsL hsr hsm+ | |
| ALS225 | MC1061 F'lacIq1Z+Y+A+ | E. Altman, University of Georgia |
| MG1665 | wt | M. Guyer et al., Quant. Biol., Cold Spring |
| | | Harbor Symp, 45, 135-140 (1981) |
| JCL 1242 | Δ(argF-lac)U169 ppc::Kn | P. Chao et al., Appl. Env. Microbiol., 59, 4261- |
| | | 4265 (1993) |
| Plasmids | Relevant Characteristics | Reference or source |
| pUC18 | Amp(R),ColE1 ori | J. Norrander et al. Gene 26 101-106 (1983) |
| pPC1 | Tet(R), pyc | M. Dunn et al., J. Bacteriol., 178, 5960-5970 |
| | | (1996) |
| pUC 18- | Amp(R), pyc regulated by Plac, | This example |
| pyc | ColE1 ori | |
| pBA11 | Cam(R), birA, P15A ori | D. Barker et al., J. Mol. Biol. 146, 469-492 |
| | | (1981) |

Construction of pUC18-pyc. The R. etli pyc gene, which encodes pyruvate carboxylase, was amplified using the polymerase chain reaction (PCR). Pfu polymerase (Stratagene, La Jolla, Calif.) was used instead of Taq polymerase and the pPC1 plasmid served as the DNA template. Primers were designed based on the published pyc gene sequence (M. Dunn et al., J. Bacteriol., 178, 5960-5970 (1996)) to convert the pyc translational start signals to match those of the lacZ gene. These primers also introduced a KpnI (GGTACC) restriction site at the beginning of the amplified fragment and a BglII (AGATCT) restriction site at the end of the amplified fragment; forward primer 5′TAC TAT GGT ACC TTA GGA AAC AGC TAT GCC CAT ATC CAA GAT ACT CGT T 3′ (SEQ ID NO: 1), reverse primer 5′ ATT CGT ACT CAG GAT CTA AAA GAT CTA ACA GCC TGA CTT TAC ACA ATC G 3′ (SEQ ID NO:2) (the KpnI, Shine Dalgarno, ATG start, and BglII sites are underlined). The resulting 3.5 kb fragment was gel isolated, restricted with KpnI and BglII and then ligated into gel isolated pUC18 DNA which had been restricted with KpnI and BamHI to form the pUC18-pyc construct. This construct, identified as “Plasmid in E. coli ALS225 pUC18-pyc” was deposited with the American Type Culture Collection (ATCC), 10801 University Blvd., Manassas, Va., 20110-2209, USA, and assigned ATCC number 207111. The deposit was received by the ATCC on Feb. 16, 1999.

Protein gels and Western blotting. Heat-denatured cell extracts were separated on 10% SDS-PAGE gels as per Altman et. al. (J. Bact., 155, 1130-1137 (1983)) and Western blots were carried out as per Carroll and Gherardini (Infect. Immun., 64, 392-398 (1996)). ALS225 E. coli cells containing either pUC18 or pUC18-pyc were grown to mid-log in rich media at 37° C. both in the presence and absence of IPTG. Because ALS225 contains lacIq1 on the F′, significant induction of the pUC18-pyc construct should not occur unless IPTG is added. Protein extracts were prepared, subjected to SDS PAGE, and Western blotted. Proteins which had been biotinylated in vivo were then detected using the Sigma-Blot protein detection kit (Sigma Chemical Corp., St. Louis, Mo.). The instructions of the manufacturer were followed except that during the development of the western blots the protein biotinylation step was omitted, thus allowing for the detection of only those proteins which had been biotinylated in vivo.

Pyruvate carboxylase (PC) enzyme assay. For pyruvate carboxylase activity measurements, 100 mL of mid-log phase culture was harvested by centrifugation at 7,000×g for 15 minutes at 4° C. and washed with 10 mL of 100 mM Tris-Cl (pH 8.0). The cells were then resuspended in 4 mL of 100 MM Tris-Cl (pH 8.0) and subsequently subjected to cell disruption by sonication. The cell debris was removed by centrifugation at 20,000×g for 15 minutes at 4° C. The pyruvate carboxylase activity was measured by the method of Payne and Morris (J. Gen. Microbiol., 59, 97-101 (1969)). In this assay the oxaloacetate produced by pyruvate carboxylase is converted to citrate by the addition of citrate synthase in the presence of acetyl CoA and 5,5-dithio-bis(2-nitro-benzoate) (DTNB) (Aldrich Chemical Co.); the homotetramer pyruvate carboxylase enzyme from R. etli requires acetyl coenzyme A for activation. The rate of increase in absorbence at 412 nm due to the presence of CoA-dependent formation of the 5-thio-2-nitrobenzoate was monitored, first after the addition of pyruvate and then after the addition of ATP. The difference between these two rates was taken as the ATP-dependent pyruvate carboxylase activity. The concentration of reaction components per milliliter of mixture was as follows: 100 mM Tris-Cl (pH 8.0), 5 mM MgCl2.H₂O, 50 mM Na HCO₃, 0.1 mM acetyl CoA, 0.25 mM DTNB, and 5 units (U) of citrate synthase. Pyruvate, ATP, ADP, or aspartate, were added as specified in the Results section, below. The reaction was started by adding 50 μl of cell extract. One unit of pyruvate carboxylase activity corresponds to the formation of 1 μmol of 5-thio-2-nitrobenzoate per mg of protein per minute. All enzyme assays were performed in triplicate and a standard error of less then 10% was observed. The total protein in the cell extracts was determined by the Lowry method (O. Lowry et al., J. Biol. Chem., 193, 265-275 (1951)).

Results

Expression of the R. etli pyruvate carboxylase enzyme in E. coli. The R. etli pyc gene, which encodes pyruvate carboxylase, was PCR amplified from pPC1 and subcloned into the pUC18 cloning/expression vector as described above. Because the translational start signals of the R. etli pyc gene were nonoptimal (pyc from R. etli uses the rare TTA start codon as well as a short spacing distance between the Shine Dalgarno and the start codon), the translational start signals were converted to match that of the lacZ gene which can be expressed at high levels in E. coli using a variety of expression vectors. When induced cell extracts of the pUC18-pyc construct were assayed via western blots developed to detect biotinylated proteins, a band of about 120 kD was detected. This value is consistent with the previously reported size assessment for the R. etli pyruvate carboxylase enzyme (M. Dunn et al., J. Bacteriol., 178, 5960-5970 (1996)). By comparing serial dilutions of the pyruvate carboxylase which was expressed from the pUC18-pyc construct with purified pyruvate carboxylase enzyme obtained commercially, it was determined that, under fully induced conditions pyruvate carboxylase from R. etli was being expressed at 1% of total cellular protein in E. coli.

Effects of biotin and biotin holoenzyme synthase on the expression of biotinylated R. etli pyruvate carboxylase in E. coli. Pyruvate carboxylase is a biotin-dependent enzyme, and mediates the formation of oxaloacetate by a two-step carboxylation of pyruvate. In the first reaction step, biotin is carboxylated with ATP and bicarbonate as substrates, while in the second reaction the carboxyl group from carboxybiotin is transferred to pyruvate. All pyruvate carboxylases studied to date have been found to be biotin-dependent and exist as multimeric proteins, but the size and structure of the associated subunits can vary considerably. Pyruvate carboxylases from different bacteria have been shown to form α₄, or α₄β₄ structures with the size of the α subunit ranging from 65 to 130 kD. In all cases, however, the α subunit of the pyruvate carboxylase enzyme has been shown to contain three catalytic domains—a biotin carboxylase domain, a transcarboxylase domain, and a biotin carboxyl carrier protein domain—which work collectively to catalyze the two-step conversion of pyruvate to oxaloacetate. In the first step, a biotin prosthetic group linked to a lysine residue is carboxylated with ATP and HCO₃⁻, while in the second step, the carboxyl group is transferred to pyruvate. The biotinylation of pyruvate carboxylase occurs post-translationally and is catalyzed by the enzyme biotin holoenzyme synthase. In this experiment, E. coli cells containing the pUC18-pyc construct were grown under inducing conditions in minimal defined media which either contained no added biotin, or biotin added at 50 or 100 ng/mL. Specifically, MG1655 pUC18-pyc cells were grown to mid-log at 37° C. in M9 media that contained varying amounts of biotin. Protein extracts were prepared, subjected to SDS PAGE, and Western blotted. Proteins which had been biotinylated in vivo were then detected using the Sigma-Blot protein detection kit, as described above. MG 1655 was used in this experiment because it grows significantly faster than ALS225 in minimal media. Because MG1655 does not contain lacIq1, maximal expression of pyruvate carboxylase could be achieved without adding IPTG. The amount of biotinylated pyruvate carboxylase that was present in each sample was quantitated using a Stratagene Eagle Eye II Still Video. The biotinylation of pyruvate carboxylase that was expressed from the pUC18-pyc construct was clearly affected by biotin levels. Cells that had to produce all their biotin de novo expressed significantly lower amounts of biotinylated protein. The addition of biotin at a final concentration of 50 ng/mL was sufficient to biotinylate all of the pyruvate carboxylase that was expressed via the pUC18-pyc construct.

Since the post-translational biotinylation of pyruvate carboxylase is carried out by the enzyme biotin holoenzyme synthase, the effect of excess biotin holoenzyme synthase on the biotinylation of pyruvate carboxylase was investigated. This analysis was accomplished by introducing the multicopy plasmid pBA11 (which contains the birA gene encoding biotin holoenzyme synthase) into E. coli cells that also harbored the pUC18-pyc construct; pBA11 is a pACYC184 derivative and thus compatible with pUC18-pyc. The effects of excess biotin holoenzyme synthase enzyme were examined in rich media where biotin would also be present in excess. Specifically, ALS225 cells containing pUC18-pyc, or pBA11 were grown to mid-log at 37° C. in rich media that contained IPTG. Protein extracts were prepared, subjected to SDS PAGE, and Western blotted, and proteins which had been biotinylated in vivo were then detected using the Sigma-Blot protein detection kit as described above. Barker et al. (J. Mol. Biol., 146, 469-492 (1981)) have shown that pBA11 causes a 12-fold increase in biotin holoenzyme synthase enzyme levels. The amount of biotinylated pyruvate carboxylase that was present in each sample was quantitated using a Stratagene Eagle Eye II Still Video System. Protein extracts prepared from cells which either contained only pUC18-pyc or both pUC18-pyc and pBA11 yielded equal amounts of biotinylated pyruvate carboxylase protein. This result suggests that a single chromosomal copy of birA is sufficient to biotinylate all of the pyruvate carboxylase that is expressed when biotin is present in excess.

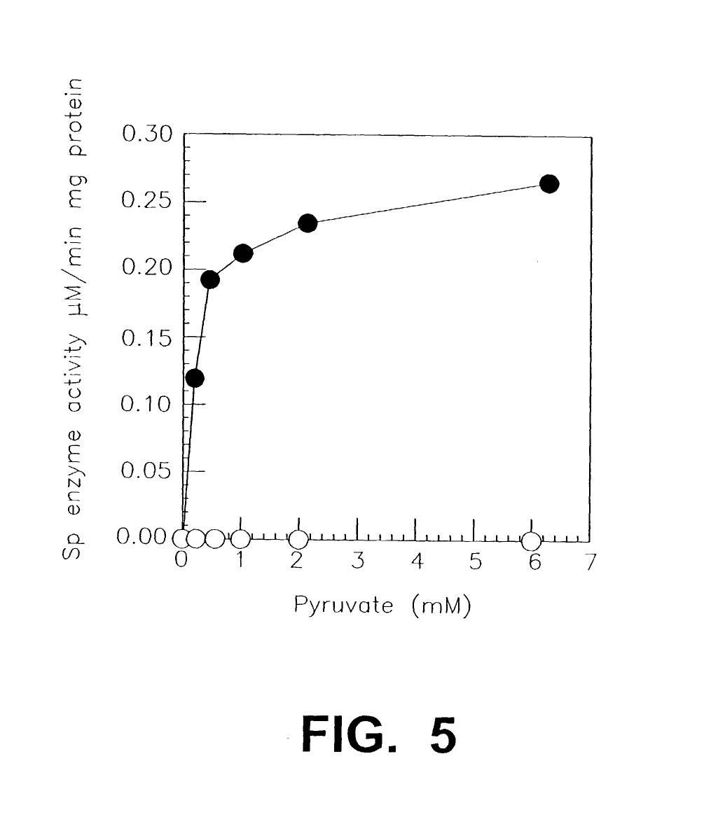

R. etli pyruvate carboxylase can convert pyruvate to oxaloacetate in E. coli. To confirm that the expressed pyruvate carboxylase protein was enzymatically active in E. coli, the coupled enzyme assay developed by Payne and Morris was employed to assess pyruvate carboxylase activity (J. Payne et al., J. Gen. Microbiol., 59, 97-101 (1969)). Cell extracts containing the induced pUC18-pyc construct (MG1655 pUC18-pyc) were tested for pyruvate carboxylase activity using varying amounts of pyruvate, and compared to controls containing the pUC18 construct (MG1655 pUC18). ATP was added at a final concentration of 5 mM to the reaction mixture and pyruvate carboxylase activity was determined in the presence of increasing amounts of pyruvate. FIG. 5 shows that E. coli cells harboring the pUC18-pyc construct could indeed convert pyruvate to oxaloacetate and that the observed pyruvate carboxylase activity followed Michaelis-Menten kinetics. A Lineweaver-Burke plot of these data revealed that the saturation constant (Kₘ) for expressed pyruvate carboxylase was 0.249 mM with respect to pyruvate. This value is in excellent agreement with other pyruvate carboxylase enzymes that have been studied (H. Feir et al., Can. J. Biochem., 47, 697-710 (1969); H. Modak et al., Microbiol., 141, 2619-2628 (1995); M. Scrutton et al., Arch. Biochem. Biophys., 164, 641-654 (1974)).

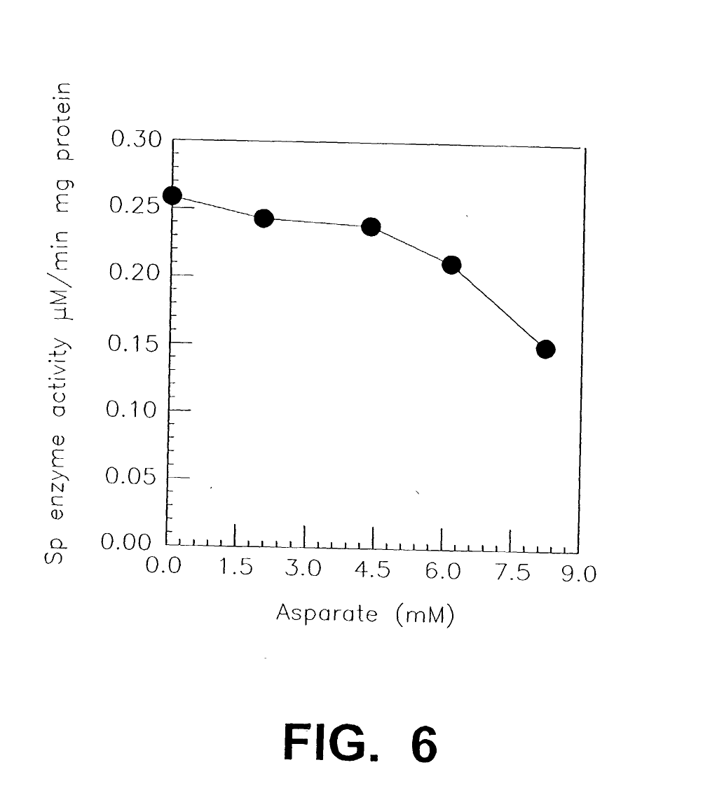

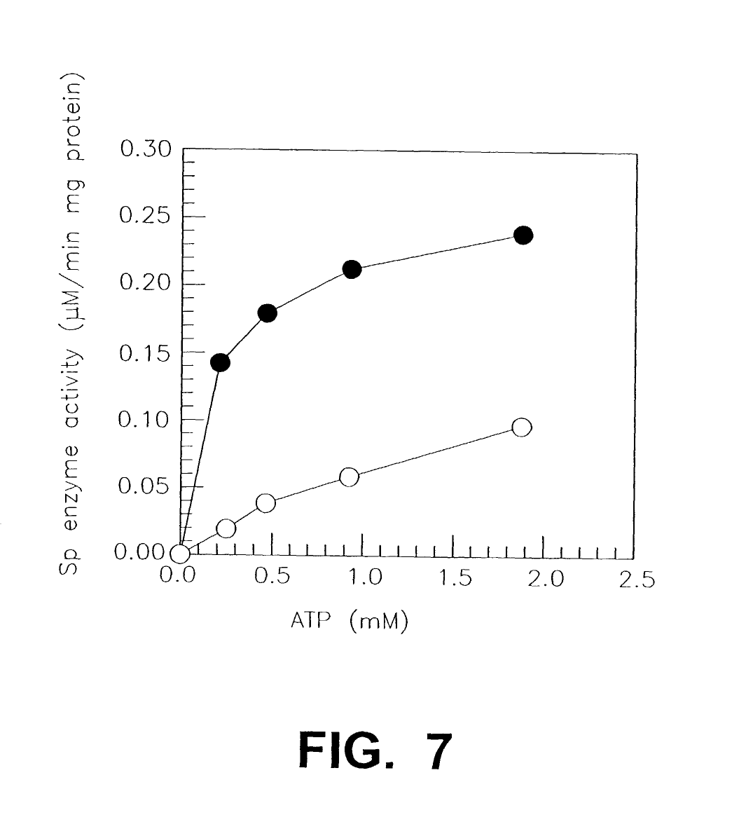

It is well documented that the α4 pyruvate carboxylase enzymes can be inhibited by either aspartate or adenosine diphosphate (ADP). Aspartate is the first amino acid that is synthesized from oxaloacetate and ADP is liberated when pyruvate carboxylase converts pyruvate to oxaloacetate. Pyruvate carboxylase activity in the presence of each of these inhibitors was evaluated using extracts of MG1655 cells that contained the pUC18-pyc construct. The effect of aspartate was analyzed by adding ATP and pyruvate to the reaction mixture to final concentrations of 5 mM and 6 mM, respectively, then determining pyruvate carboxylase activity in the presence of increasing amounts of aspartate. FIG. 6 shows the pyruvate carboxylase activity that was obtained in the presence of different concentrations of aspartate. As expected, the pyruvate carboxylase activity was inhibited by aspartate and the specific activity decreased to approximately 43% in the presence of 8 mM aspartate. The effect of ADP was analyzed by adding pyruvate to the reaction mixture to a final concentration of 5 mM, then determining pyruvate carboxylase activity in the presence of increasing amounts of ATP. FIG. 7 shows that ADP severely affected the observed pyruvate carboxylase activity and acted as a competitive inhibitor of ATP. A Lineweaver-Burke plot of these data revealed that the saturation constant (Kₘ) for expressed pyruvate carboxylase was 0.193 mM with respect to ATP and that the inhibition constant for ADP was 0.142 mM. Again, these values were in excellent agreement with other pyruvate carboxylase enzymes that have been studied H. Feir et al., Can. J. Biochem., 47, 697-710 (1969); H. Modak et al., Microbiol., 141, 2619-2628 (1995); M. Scrutton et al., Arch. Biochem. Biophys., 164, 641-654 (1974)).

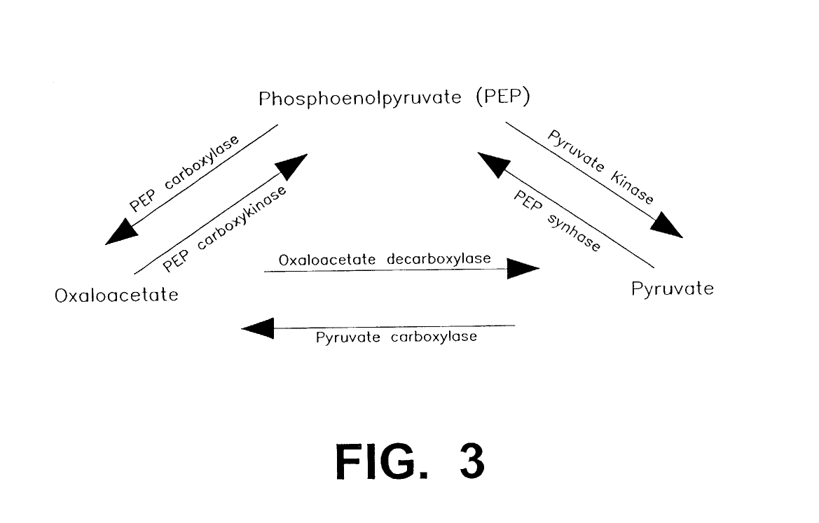

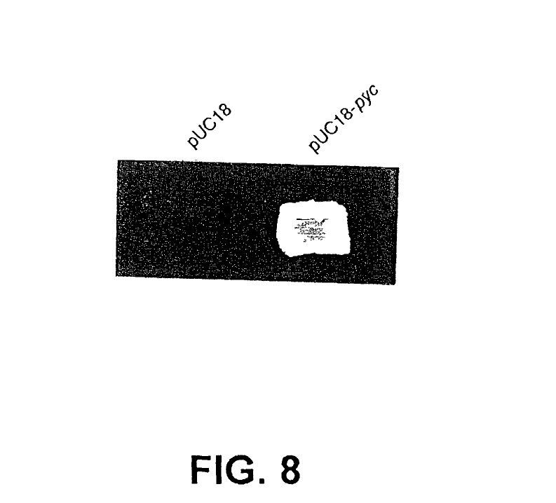

To show that the expression of R. etli pyruvate carboxylase in E. coli can truly divert carbon flow from pyruvate to oxaloacetate, we tested whether the pUC18-pyc construct could enable an E. coli strain which contained a ppc null allele (ppc encodes PEP carboxylase) to grow on minimal glucose media. Because E. coli lacks pyruvate carboxylase and thus is only able to synthesize oxaloacetate from PEP, (see FIG. 3) E. coli strains which contain a disrupted ppc gene can not grow on minimal media which utilizes glucose as the sole carbon source (P. Chao et al., Appl. Env. Microbiol., 59, 4261-4265 (1993)). The cell line used for this experiment was JCL1242 (ppc::kan), which contains a kanamycin resistant cassette that has been inserted into the ppc gene and thus does not express the PEP carboxylase enzyme. JCL1242 cells containing either pUC18 or the pUC18-pyc construct were patched onto minimal M9 glucose thiamine ampicillin IPTG plates and incubated at 37° C. for 48 hours. As shown in FIG. 8, E. coli cells which contain both the ppc null allele and the pUC18-pyc construct were able to grow on minimal glucose plates. This complementation demonstrates that a branch point can be created at the level of pyruvate which results in the rerouting of carbon flow towards oxaloacetate, and clearly shows that pyruvate carboxylase is able to divert carbon flow from pyruvate to oxaloacetate in E. coli.

### EXAMPLE II

### Expression of R. etli Pyruvate Carboxylase Causes Increased Succinate Production in E. coli

Materials and Methods

Bacterial strains and plasmids. The E. coli strains used in this study are listed in Table 2. The lactate dehydrogenase mutant strain designated RE02 was derived from MG1655 by P1 phage transduction using E. coli strain NZN111 (P. Bunch et al., Microbiol., 143, 187-195 (1997)).

TABLE 2
Strains and plasmids used.

| Strains | Genotype | Reference or Source |
|---|---|---|
| MG1655 | Wild type | M. Guyer et al., Quant. |
| | | Biol, Cold Spring |
| | | Harbor Symp., 45, |
| | | 135-140 (1981) |
| RE02 | MG1655 ldh | This example |
| Plasmids | Relevant Characteristics | Reference or Source |
| pUC18-pyc | Amp(R), pyc regulated by Plac | Example I |
| pTrc99A | Amp(R), lacIq, Ptrc | E. Amann et al., Gene, |
| | | 69:301-315 (1988) |
| pTrc99A-pyc | Amp(R), lacIq, pyc regulated by | This example |
| | Ptrc | |

The pyc gene from R. etli was originally cloned under the control of the lac promoter (Example I). Because this promoter is subjected to catabolic repression in the presence of glucose, a 3.5 kb XbaI-KpnI fragment from pUC18-pyc was ligated into the pTrc99A expression vector which had been digested with XbaI and KpnI. The new plasmid was designated as pTrc99A-pyc. This plasmid, identified as “Plasmid in E. coli ALS225 pTrc99A-pyc”, was deposited with the American Type Culture Collection (ATCC), 10801 University Blvd., Manassas, Va., 20110-2209, USA, and assigned ATCC number 207112. The deposit was received by the ATCC on Feb. 16, 1999. In this new construct the transcription of the pyc gene is under the control of artificial trc promoter and thus is not subjected to catabolic repression in the presence of glucose.

Media and growth conditions. For strain construction, E. coli strains were grown aerobically in Luria-Bertani (LB) medium. Anaerobic fermentations were carried out in 100 mL serum bottles with 50 mL LB medium supplemented with 20 g/L glucose and 40 g/L MgCO₃. The fermentations were terminated at 24 hours at which point the pH values of all fermentations were approximately pH 6.7, and glucose was completely utilized. For plasmid-containing strains either ampicillin or carbenicillin was added to introduce selective pressure during the fermentation. Each of these antibiotics was introduced initially at 100 μg/mL. In one set of experiments, no additional antibiotic was added during fermentation, while in a second set of experiments an additional 50 μg/mL was added at 7 hours and 14 hours. Pyruvate carboxylase was induced by adding 1 mM IPTG. For enzyme assays cells were grown in LB medium supplemented with 20 g/L glucose and buffered with 3.2 g/L Na₂CO₃.

Fermentation product analysis and enzyme assays. Glucose, succinate, acetate, formate, lactate, pyruvate and ethanol were analyzed by high-pressure liquid chromatography (HPLC) using a Coregel 64-H ion-exclusion column (Interactive Chromatography, San Jose, Calif.) and a differential refractive index detector (Model 410, Waters, Milford, Mass.). The eluant was 4 mN H2SO4 and the column was maintained at 60° C.

For enzyme activity measurements, 50 mL of mid-log phase culture were harvested by centrifugation (10000×g for 10 minutes at 4° C.) and washed with 10 mL of 100 mM Tris-HCl buffer (pH 8.0). The cells were then resuspended in 2 mL of 100 mM Tris-HCl buffer and subjected to cell disruption by sonication. Cell debris were removed by centrifugation (20000×g for 15 minutes at 4° C.). Pyruvate carboxylase activity (J. Payne et al., J. Gen. Microbiol. 59, 97-101 (1969); see also Example I), and the endogenous activities of PEP carboxylase (K. Terada et al., J. Biochem., 109, 49-54 (1991)), malate dehydrogenase and lactate dehydrogenase (P. Bunch et al., Microbiol., 143, 187-195 (1997)) were then measured. The total protein in the cell extract was determined using the Lowry method.

Results

Table 3 shows that pyruvate carboxylase activity could be detected when the pTrc99A-pyc construct was introduced into either wild type cells (MG1655) or wild type cells which contained a ldh⁻ null mutation (RE02). The presence of IPTG did not significantly affect the expression of other important metabolic enzymes such as PEP carboxylase, lactate dehydrogenase and malate dehydrogenase.

TABLE 3
Enzyme activity in exponential phase cultures.

| | | | Specific activity | | |
|---|---|---|---|---|---|
| | | | (μmol/min mg protein) | | |
| | | | | Lactate | Malate |
| | | Pyruvate | PEP | de- | de- |
| | | carbox- | carbox- | hydro- | hydro- |
| Strain | IPTG | ylase | ylase | genase | genase |
| MG1655 | − | 0.00 | 0.15 | 0.31 | 0.06 |
| | + | 0.00 | 0.18 | 0.38 | 0.06 |
| MG1655 pTrc99A-pyc | − | 0.00 | 0.15 | 0.32 | 0.05 |
| | + | 0.22 | 0.11 | 0.32 | 0.05 |
| RE02 | − | 0.00 | 0.15 | 0.00 | 0.04 |
| | + | 0.00 | 0.13 | 0.00 | 0.04 |
| RE02 pTrc99A-pyc | − | 0.00 | 0.15 | 0.00 | 0.04 |
| | + | 0.32 | 0.12 | 0.00 | 0.05 |

In order to elucidate the effect of pyruvate carboxylase expression on the distribution of the fermentation end products, several 50 mL serum bottle fermentations were conducted (see Table 4).

TABLE 4
Effect of pyruvate carboxylase on product distribution from E. coli glucose fermentation.

| | | Mode of | | | | | | |
|---|---|---|---|---|---|---|---|---|
| | | antibiotic | Pyruvate | Succinate | Lactate | Formate | Acetate | Ethanol |
| Strain | Antibiotic | addition₁₃ | (g/L) | (g/L) | (g/L) | (g/L) | (g/L) | (g/L) |
| MG1655 (wt) | — | — | 0.00 (0.00) | 1.57 (0.17) | 4.30 (0.73) | 4.34 (0.50) | 3.34 (0.36) | 2.43 (0.24) |
| MG1655 pTrc99A-pyc | Amp | 1x | 0.00 (0.00) | 4.36 (0.45) | 2.22 (0.49) | 3.05 (0.57) | 3.51 (0.03) | 2.27 (0.30) |
| MG1655 pTrc99A-pyc | Car | 1x | 0.00 (0.00) | 4.42 (0.44) | 2.38 (0.76) | 2.94 (0.46) | 3.11 (0.36) | 2.27 (0.36) |
| MG1655 pTrc99A-pyc | Amp | 3x | 0.00 (0.00) | 4.41 (0.07) | 1.65 (0.08) | 4.17 (0.15) | 3.93 (0.11) | 2.91 (0.34) |
| MG1655 pTrc99A-pyc | Car | 3x | 0.00 (0.00) | 4.37 (0.06) | 1.84 (0.07) | 4.09 (0.08) | 3.88 (0.06) | 2.58 (0.09) |
| RE02 (ldh) | — | — | 0.61 (0.06) | 1,73 (0.12) | 0.00 (0.00) | 6.37 (0.46) | 4.12 (0.30) | 3.10 (0.26) |
| RE02 pTrc99A-pyc | Amp | 1x | 0.33 (0.11) | 2.92 (0.12) | 0.00 (0.00) | 5.38 (0.12) | 4.09 (0.16) | 2.53 (0.03) |
| RE02 pTrc99A-pyc | Car | 1x | 0.25 (0.05) | 2.99 (0.55) | 0.00 (0.00) | 5.50 (0.90) | 4.23 (0.71) | 2.50 (0.44) |
| RE02 pTrc99A-pyc | Amp | 3x | 0.30 (0.04) | 2.74 (0.07) | 0.00 (0.00) | 6.48 (0.04) | 4.75 (0.06) | 2.99 (0.03) |
| RE02 pTrc99A-pyc | Car | 3x | 0.33 (0.04) | 2.65 (0.05) | 0.00 (0.00) | 6.21 (0.18) | 4.60 (0.12) | 3.05 (0.07) |

Antibiotics were either added once at 0 hours at a concentration of 100 μg/mL (1×) or added at 0 hours at a concentration of 100 μg/mL and again at 7 hours and 14 hours at 50 μg/L (3×). Values are the mean of three replicates and standard deviations are shown in parentheses. To calculate the net yield of each product per gram of glucose consumed, the final product concentration is divided by 20 g/L of glucose.

As shown in Table 4, expression of pyruvate carboxylase caused a significant increase in succinate production in both MG1655 (wild type) and RE02 (ldh⁻). With MG1655 the induction of pyruvate carboxylase increased the production of succinate 2.7-fold from 1.57 g/L in the control strain to 4.36 g/L, thus making succinate the major product of glucose fermentation. This increase in succinate was accompanied by decreased lactate and formate formation, indicating that carbon was diverted away from lactate toward succinate formation. A similar carbon diversion from lactate toward succinate was achieved previously by the overexpression of native PEP carboxylase (C. Millard et al., Appl. Environ. Microbiol., 62, 1808-1810 (1996)). Table 4 also shows that ampicillin and carbenicillin were equally effective in maintaining sufficient selective pressure, and that the addition of more of either antibiotic during the fermentation did not further enhance the succinate production. This evidence indicates that an initial dose (of 100 μg/mL) is sufficient to maintain selective pressure throughout the fermentation, a result which might be due to the relatively high final pH (6.8) observed in our fermentation studies versus the final pH (6.0) observed in previous studies (C. Millard et al., Appl. Environ. Microbiol., 62, 1808-1810 (1996)).

Because introducing pyruvate carboxylase into E. coli was so successful at directing more carbon to the succinate branch, we were also interested in determining whether additional carbon could be directed to succinate by eliminating lactate dehydrogenase, since this enzyme also competes for pyruvate. Table 4 compares the results of fermentations using the RE02 (ldh⁻) strain with or without the pTrc99A-pyc plasmid. Compared to the wild type strain (MG1655), the RE02 strain showed no significant change in succinate production. Instead, fermentations with the RE02 strain, whether it contained the pTrc99A-pyc plasmid or not, resulted in increased formate, acetate and ethanol production, accompanied by secretion of pyruvate. The fact that pyruvate was secreted into the fermentation broth indicates that the rate of glycolysis was greater than the rate of pyruvate utilization. The observed increase in formate concentrations in the ldh⁻ mutant may be caused by the accumulation of pyruvate, a compound which is known to exert a positive allosteric effect on pyruvate formate lyase (G. Sawers et al., J. Bacteriol., 170, 5330-5336 (1988)). With RE02 the induction of pyruvate carboxylase increased the production of succinate 1.7-fold from 1.73 g/L in the control strain to 2.92 g/L. Thus, the succinate increase obtained in the ldh⁻ mutant strains was significantly lower than that obtained in the wild type strain (MG1655). A possible explanation for this observation might be that pyruvate carboxylase activity was inhibited by a cellular compound which accumulated in the ldh⁻ mutants.

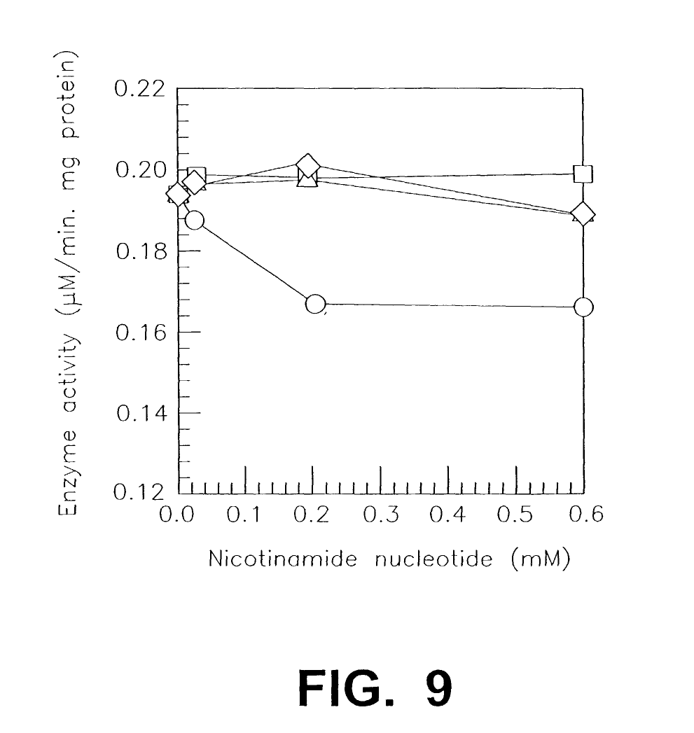

During glycolysis two moles of reduced nicotinamide adenine dinucleotide (NADH) are generated per mole of glucose. NADH is then oxidized during the formation of ethanol, lactate and succinate under anaerobic conditions. The inability of the ldh⁻ mutants to consume NADH through lactate formation may put stress on the oxidizing capacity of these strains, leading to an accumulation of NADH. Indeed, this reduced cofactor has previously been shown to inhibit a pyruvate carboxylase isolated from Saccharomyces cerevisiae (J. Cazzulo et al., Biochem. J., 112, 755-762 (1969)). In order to elucidate whether such oxidizing stress might be the cause of the attenuated benefit that was observed when pyruvate carboxylase was expressed in the ldh⁻ mutants, we investigated the effect of both oxidized and reduced nicotinamide adenine dinucleotide (NADH/NAD+) and dinucleotide phosphate (NADPH/NADP+) on pyruvate carboxylase activity. Enzyme assays were conducted with cell-free crude extract obtained from MG1655 pTrc99A-pyc. All assays were conducted in triplicate, and average values are shown in FIG. 9. Standard deviation was no greater than 5% for all data points. NADH inhibited pyruvate carboxylase, whereas NAD+, NADP+ and NADPH did not. The lower succinate enhancement with RE02 the ldh⁻ mutant is therefore hypothesized to result from an accumulation of intracellular NADH, a cofactor which appears to inhibit pyruvate carboxylase activity.

### EXAMPLE III

### Expression of R. etli Pyruvate Carboxylase Does Not Affect Glucose Uptake in E. coli in Anaerobic Fermentation

Methods

Microorganisms and plasmids. E. coli strain MG1655 (wild type F⁻λ⁻; M. Guyer et al., Quant. Biol., Cold Spring Harbor Symp., 45, 135-140 (1981); see also Example I) and the plasmid pUC18-pyc which contains the pyc gene from R. etli (see Example I).

Media and fermentation. All 2.0 L fermentations were carried out in 2.5 L New Brunswick Baffle III bench top fermenters (New Brunswick Scientific, Edison, N.J.) in Luria-Bertani (LB) supplemented with glucose, 10 g/L; Na₂PHO₄.7H₂O, 3 g/L; KH₂PO₄, 1.5 g/L; NH₄Cl, 1 g/L; MgSO₄.7H₂O, 0.25 g/L; and CaCl₂.2H₂O, 0.02 g/L. The fermenters were inoculated with 50 mL of anaerobically grown culture. The fermenters were operated at 150 rpm, 0% oxygen saturation (Ingold polarographic oxygen sensor, New Brunswick Scientific, Edison, N.J.), 37° C., and pH 6.4, which was controlled with 10% NaOH. Anaerobic conditions were maintained by flushing the headspace of the fermenter with oxygen-free carbon dioxide. When necessary, the media was supplemented with an initial concentration of 100 μg/mL ampicillin, previously shown to be sufficient to maintain the selective pressure (Example I).

Analytical methods. Cell growth was monitored by measuring the optical density (OD) (DU-650 spectrophotometer, Beckman Instruments, San Jose, Calif.) at 600 nm. This optical density was correlated with dry cell mass using a calibration curve of dry cell mass (g/L)=0.48×OD. Glucose and fermentation products were analyzed by high-pressure liquid chromatography using Coregel 64-H ion-exclusion column (interactive Chromatography, San Jose, Calif.) as described in Example II.

The activity of pyruvate carboxylase and the endogenous activity of PEP carboxylase was measured by growing each strain and clone separately in 160 mL serum bottles under strict anaerobic conditions. Cultures were harvested in mid-logarithmic growth, washed and subjected to cell disruption by sonication. Cell debris were removed by centrifugation (20000×g for 15 min at 4° C.). Pyruvate carboxylase activity was measured as previously described (Payne and Morris, 1969), and the PEP carboxylase activity was measured in the absence of ATP using PEP instead of pyruvate as the substrate, with the appearance of CoA-dependent thionitrobenzoate formation at 412 nm monitored. The total protein in the cell extract was determined using the Lowry method.

Results

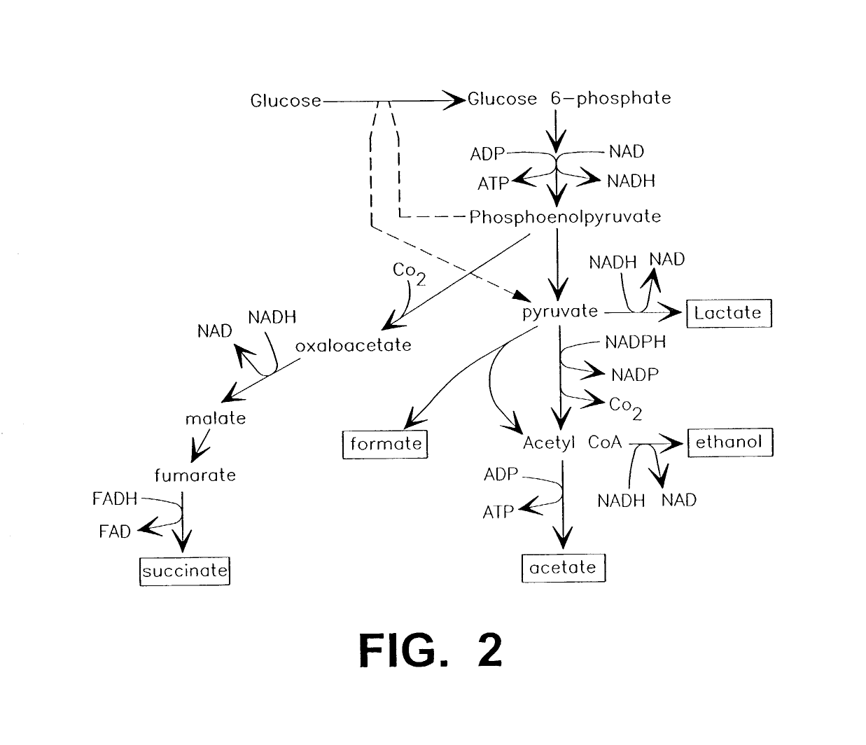

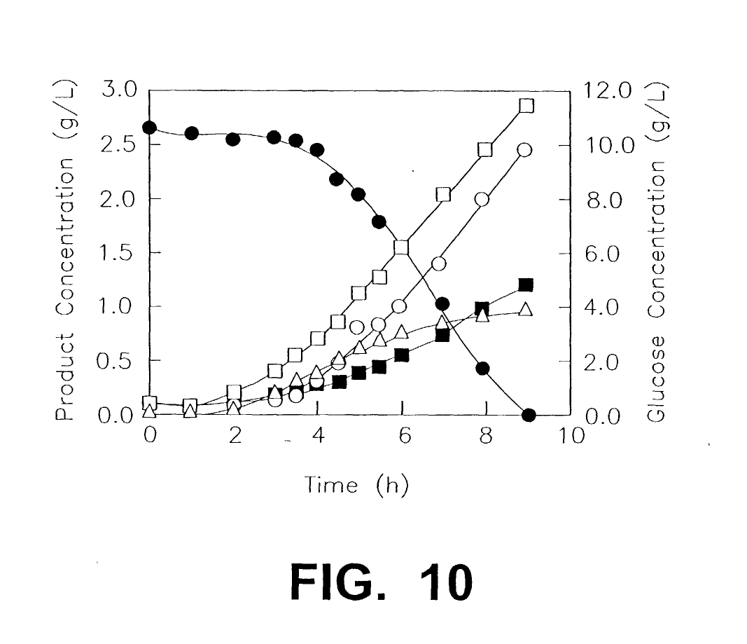

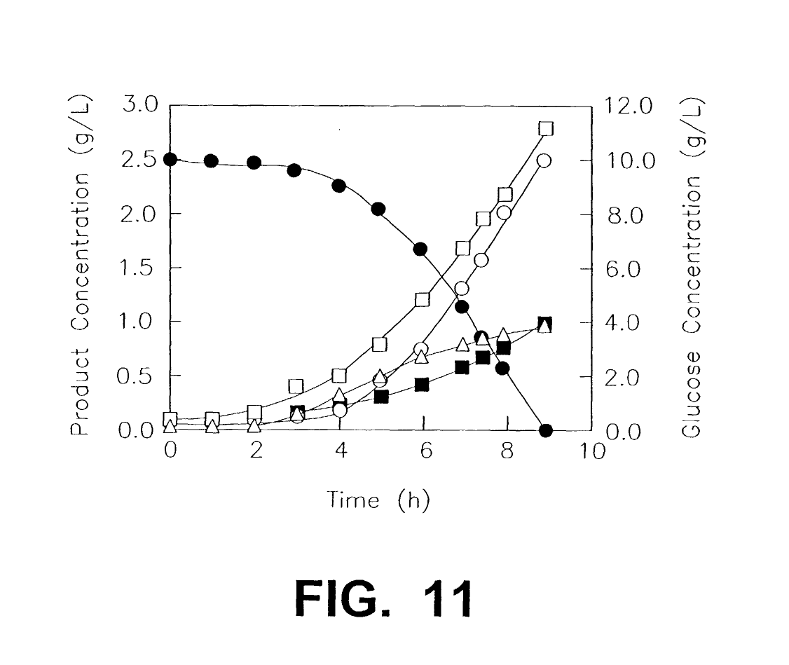

E. coli MG1655 grew anaerobically with 10 g/L glucose as energy and carbon source to produce the end products shown in FIG. 2. The participation of phosphoenolpyruvate in glucose uptake is shown by the dashed line. The biochemical pathway is not stoichiometric nor are all cofactors shown. FIG. 10 shows the dry cell mass, succinate, lactate, formate and glucose concentrations with time in a typical 2-liter fermentation of this wild-type strain. FIG. 11 shows these concentrations with time in a fermentation of this wild-type strain with the cloning/expression vector pUC18. After complete glucose utilization, the average final concentration of succinate for the wild-type strain was 1.18 g/L, while for the wild-type strain with the vector pUC18 the final succinate concentration was 1.00 g/L. For these fermentations, the average final lactate concentration was 2.33 g/L for the wild-type strain and 2.27 g/L for the same strain with pUC18.

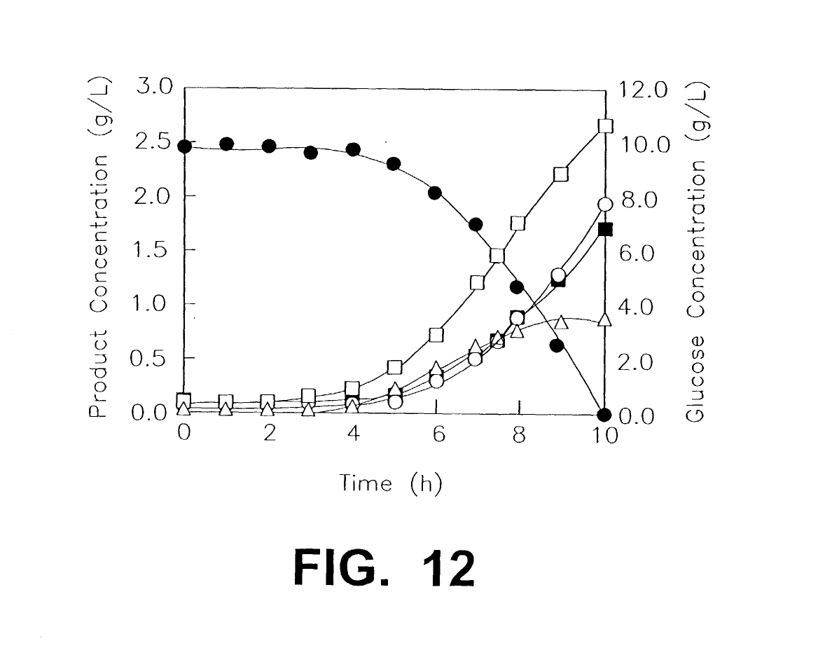

FIG. 12 shows the concentrations with time of dry cell mass, succinate, lactate, formate and glucose in a fermentation of the strain containing the pUC18-pyc plasmid. This figure shows that the expression of pyruvate carboxylase causes a substantial increase in final succinate concentration and a decrease in lactate concentration. Specifically, for the wild-type with pUC18-pyc the average final succinate concentration was 1.77 g/L, while the average final lactate concentration was 1.88 g/L. These concentrations correspond to a 50% increase in succinate and about a 20% decrease in lactate concentration, indicating that carbon was diverted from lactate toward succinate formation in the presence of the pyruvate carboxylase.

The activities of PEP carboxylase and pyruvate carboxylase were assayed in cell-free extracts of the wild type and the plasmid-containing strains, and these results are shown in Table 5. In the wild type strain and the strain carrying the vector no pyruvate carboxylase activity was detected, while this activity was detected in MG1655/pUC18-pyc clone. PEP carboxylase activity was observed in all three strains.

TABLE 5
Enzyme activity in mid-logarithmic growth culture.

Sp. activity (μmol/min mg protein)

| | Pyruvate | PEP |
|---|---|---|
| Strain | carboxylase | carboxylase |
| MG1655 | 0.0 | 0.10 |
| MG1655/pUC18 | 0.0 | 0.12 |
| MG1655/pUC18-pyc | 0.06 | 0.08 |

To determine the rates of glucose consumption, succinate production, and cell mass production during the fermentations, each set of concentration data was regressed to a fifth-order polynomial. (These best-fitting curves are shown in FIGS. 10-12 with the measured concentrations.) By taking the first derivative of this function with respect to time, an equation results which provides these rates as functions of time. This procedure is analogous to previous methods (E. Papoutsakis et al., Biotechnol. Bioeng, 27, 50-66 (1985); K. Reardon et al., Biotechnol. Prog, 3, 153-167 (1987)) used to calculate metabolic fluxes. In the case of fermentations with both pyruvate carboxylase and PEP carboxylase present, however, the flux analysis cannot be completed due to a mathematical singularity at the PEP/pyruvate nodes (S. Park et al., Biotechnol. Bioeng, 55, 864-B879 (1997)). Nevertheless, using this approach the glucose uptake and the rates of succinate and cell mass production may be determined.

TABLE 6
Rates of glucose uptake, succinate production, and cell production.

| Parameter | MG1655 | MG1655/pUC18 | MG1655/pUC18-pyc |
|---|---|---|---|
| Glucose uptake (maximum) | 2.17 (0.10) | 2.40 (0.01) | 2.47 (0.01) |
| Glucose uptake (average during final 4 | 1.99 (0.05) | 2.00 (0.06) | 1.99 (0.05) |
| hours of fermentations) | | | |
| Rate of succinate production (at time of | 0.234 (0.010) | 0.200 (0.012) | 0.426 (0.015) |
| max. glucose uptake) | | | |
| Rate of succinate production (average | 0.207 (0.005) | 0.177 (0.009) | 0.347 (0.002) |
| during final 4 hours) | | | |
| Cell production (maximum) | 0.213 (0.006) | 0.169 (0.033) | 0.199 (0.000) |

Table 6 shows the results of calculating the rates of glucose uptake, and succinate and cell mass production in a wild-type E. coli strain (MG1655), the wild-type strain with the pUC18 cloning/expression vector (MG1655/pUC18) and the wild-type strain with MG1655/pUC18-pyc. All units are g/Lh, and the values in parentheses represent standard deviation of measurements.

As these results demonstrate, the addition of the cloning vector or the vector with the pyc gene had no significant effect on the average glucose uptake during the final 4 hours of the fermentations. Indeed, the presence of the pyc gene actually increased the maximum glucose uptake about 14% from 2.17 g/Lh to 2.47 g/Lh. The presence of the pUC18 cloning vector reduced slightly the rates of succinate production. As expected from the data shown in FIG. 12, the expression of the pyc gene resulted in an 82% increase in succinate production at the time of maximum glucose uptake, and a 68% increase in the rate of succinate production during the final 4 hours of the fermentations. The maximum rate of cell growth (which occurred at 4-5 hours for each of the fermentations) was 0.213 g/Lh in the wild type strain, but decreased in the presence of pUC18 (0.169 g/Lh) or pUC18-pyc (0.199 g/Lh). Similarly, the overall cell yield was 0.0946 g dry cells/g glucose consumed for the wild-type, but 0.0895 g/g for the wild-type with pUC18 and 0.0882 g/g for the wild-type strain with pUC18-pyc. This decrease in biomass may be due to the expenditure of one mole of energy unit (ATP) per mole of pyruvate converted to oxaloacetate by pyruvate carboxylase and the increased demands of protein synthesis in the plasmid-containing strains. A specific cell growth rate could not be calculated since the growth of this strain shows logarithmic growth only for the first few hours of growth. In summary, expression of pyruvate carboxylase from R. etli in E. coli causes a significant increase in succinate production at the expense of lactate production without affecting glucose uptake. This result has dramatic ramifications for bacterial fermentation processes which are used to produce oxaloacetate-derived biochemicals. Because overexpress ion of pyruvate carboxylase causes increased production of oxaloacetate-derived biochemicals without affecting glucose uptake, this technology can be advantageously employed in fermentation processes in order to obtain more product per amount of inputted glucose.

### EXAMPLE IV

### Expression of R. etli Pyruvate Carboxylase Causes Increased Threonine Production in E. coli

Materials and Methods

Bacterial strains and plasmids. The threonine-producing strain βIM-4 (ATCC 21277) was used in this study (Shiio and Nakamori, Agr. Biol. Chem., 33, 1152-1160 (1969); I. Shiio et al. U.S. Pat. No. 3,580,810 (1971)). This strain was transformed with either pTrc99A-pyc (see Example II) or pTrc99A (E. Amann et al., Gene, 69, 301-315 (1988)).

Media and growth conditions. Aerobic fermentations were carried out in 2.0 L volume in Bioflow II Fermenters. The media used for these fermentation contained (per liter): glucose, 30.0 g; (NH₄)₂SO₄ 10.0 g, FeSO₄.H₂O, 10.0 mg; MnSO₄.H₂O, 5.5 mg/L; L-proline, 300 mg; L-isoleucine, 100 mg; L-methionine, 100 mg; MgSO₄.7H₂O, 1 g; KH₂PO₄, 1 g; CaCO₃, 20 g; thiamine.HCl, 1 mg; d-biotin, 1 mg. In order to maintain selective pressure for the plasmid-carrying strains, media were supplemented initially with 50 mg/L ampicillin. Also, IPTG was added to a final concentration of 1 mmol/L at 2 hours to fermentations performed with either of these strains.

Fermentation product analysis. Cell growth was determined by measuring optical density at 550 nm of a 1:21 dilution of sample in 0.1M HCl. Glucose, acetic acid and other organic acids were analyzed by high-pressure liquid chromatography as previously described (Eiteman and Chastain, Anal. Chim. Acta, 338, 69-75 (1997)) using a Coregel 64-H ion-exclusion column. Threonine was quantified by high-pressure liquid chromatography using the ortho-phthaldialdehyde derivatization method (D. Hill, et al., Anal. Chem., 51, 1338-1341 (1979); V. Svedas, et al. Anal. Biochem., 101, 188-195 (1980)).

Results

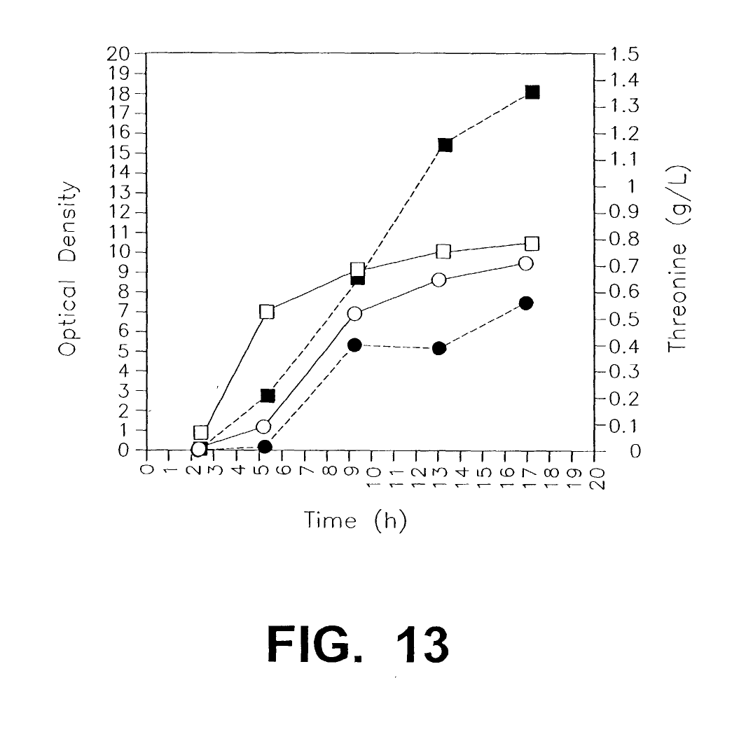

The threonine-producing strain βIM-4 (ATCC 21277), harboring either the control plasmid pTrc99A or the plasmid pTrc99A-pyc which overproduces pyruvate carboxylase, was grown aerobically with 30 g/L glucose as energy and carbon source and the production of threonine was measured. As shown in FIG. 13, the overproduction of pyruvate carboxylase caused a significant increase in the production of threonine in the threonine-producing E. coli strain. At 17 hours when the initial inputted glucose had been consumed, a concentration of 0.57 g/L threonine was detected in the parental strain harboring the pTrc99A control plasmid, while a concentration of 1.37 g/L threonine was detected in the parental strain harboring the pTrc99A-pyc plasmid. Given that the final OD₅₅₀ of both cultures were within 10% of each other at the end of the fermentation, the 240% increase in threonine concentration caused by the overproduction of pyruvate carboxylase can be deemed to be significant. As in our anaerobic fermentation studies (see Example III), we found that glucose uptake was not adversely affected by the overproduction of pyruvate carboxylase.

### EXAMPLE V

### Expression of R. etli Pyruvate Carboxylase from an E. coli/C. Glutamicum Shuttle Vector

The E. coli/C. glutamicum shuttle vector pEKEX1 allows genes to be overexpressed in both E. coli and in C. glutamicum. Unfortunately, however, it only contains four restriction sites, EcoRI, BamHI, SalI and PstI, that can be used for cloning, three of which are already present in the R. etli pyc gene. For this reason, a derivative vector, pEKEX1A, was constructed which introduced a KpnI cloning site between the EcoRI and BamHI sites and a BglII cloning site between the BamHI and SalI sites. The following two oligonucleotides, 5′ AAT TCG GTA CCG GAT CCA GAT CTG 3′ (SEQ ID NO: 1) and 5′TCG ACA GAT CTG GAT CCG GTA CCG 3′ (SEQ ID NO:2), which were phosphorylated at their 5′ ends, were annealed and ligated into the pEKEX1 vector which had been digested with BamHI and HindIII to create pEKEX1A. Restriction analysis was then performed to ensure that all the cloning sites were present in the new vector as expected. To construct pEKEX1A-pyc, a 3.5 kb KpnI, SalI fragment from pUC18-pyc that contained the pyc gene was ligated into the pEKEX1A vector which had been digested with the same to restriction enzymes. Successful expression from pEKEX1A was demonstrated in E. coli ALS225. Pyruvate carboxylase was detected via Western Blot analysis and the Payne and Morris pyruvate carboxylase activity assay. Because of the successful expression from the shuttle vector in E. coli, it is expected that an exogenous pyc gene can likewise be introduced into C. glutamicum to increase expression levels of pyruvate carboxylase in C. glutamicum as well.

### EXAMPLE VI

### Enhanced Synthesis of Lysine by C. glutamicum

C. glutamicum has long been the preferred microorganism for enzymatic production of lysine in the biochemicals industry. Naturally occurring strains of C. glutamicum make more of the oxaloacetate derived amino acids than many other known microbes. See Kroschwitz et al., eds., Encyclopedia of Chemical Technology, 4th Ed., Vol. 2, pp. 534-570 (1992). Strains that are used commercially to make lysine are typically those wherein all biosynthetic branches after oxaloacetate which make any amino acid other than lysine have been knocked out, thus maximizing the biosynthesis of lysine. The enzyme pyruvate carboxylase has only recently been found in C. glutamicum, and it does not appear to be highly expressed when C. glutamicum is grown on media which uses glucose as the carbon source (P. Peters-Wendisch et al., Microbiology (Reading), 143, 1095-1103 (1997); M. Koffas et al., GenBank submission number AF038548 (submitted Dec. 14, 1997). Although it contains its own endogenous pyruvate carboxylase, a more convenient way to overexpress this enzyme in C. glutamicum is to insert the foreign gene pyc from R. etli. Accordingly, the current construct from pUC18 as described in Examples I and II will be transferred into C. glutamicum using the shuttle vector pEXO (G. Eikmanns et al., Gene, 102, 93-98 (1991)). Overexpression of pyruvate carboxylase in Corynebacterium glutamicum can also be achieved using the gene encoding pyruvate carboxylase from P. fluorescens. Carbon is expected to be diverted to lysine in an aerobic fermentation and increase lysine yield.

### EXAMPLE VII

### Enhanced Synthesis of Lysine by C. glutamicum Auxotrophs

Recent evidence demonstrates that acetate, valine and alanine each accumulate in the latter stages of lysine synthesis in C. glutamicum (J. Vallino et al., Biotechnol. Bioeng., 41, 633-646 (1993)). Since each of these products is derived directly from pyruvate, this observation suggests that a bottleneck exists in the pathway at pyruvate (see FIG. 1). C. glutamicum that has been engineered according to the invention to overexpress pyruvate carboxylase already has an additional means of consuming pyruvate, and even more carbon can be diverted to lysine if one or more of these pathways are blocked. Alanine and valine auxotrophs and acetate-mutants of C. glutamicum can be engineered to overexpress pyruvate carboxylase according to the invention, to further enhance lysine yield.

### EXAMPLE VIII

### Enhanced Synthesis of Threonine in C. glutamicum

C. glutamicum can also be used to produce threonine, however, the strains that are used for the synthesis of threonine are different from the strains that are used for the synthesis of lysine. In the threonine-producing strains, all biosynthetic branches after oxaloacetate which make any amino acid other than threonine have been knocked out, thus maximizing the biosynthesis of threonine. Since the difference between lysine-producing and threonine-producing strains occurs after the oxaloacetate node, the metabolic engineering technology of the invention can equally be applied to the threonine-producing strains of C. glutamicum to enhance threonine synthesis. Synthesis of threonine is further enhanced in a C. glutamicum auxotroph as described above with in Example VI relating to lysine synthesis in C. glutamicum.

### EXAMPLE IX

### Enhancement of Biochemical Production Using Pyruvate Carboxylase from P. fluorescens

One of the main reasons the metabolic network responsible for regulating the intracellular levels of oxaloacetate is so tightly controlled is due to the fact that the key enzymes which are involved in this process are both positively and negatively regulated. In most organisms such as R. etli, pyruvate carboxylase requires the positive effector molecule acetyl coenzyme A for its activation and is repressed due to feedback inhibition by aspartate (P. Attwood, Intl. J. Biochem. Cell Biol., 27, 231-249 (1995); M. Dunn et al., J. Bacteriol., 178, 5960-5970 (1996)). The benefits obtained from overproducing R. etli pyruvate carboxylase are thus limited by the fact that diverting carbon from pyruvate to oxaloacetate both depletes acetyl coenzyme A levels and increases aspartate levels. The pyruvate carboxylase from P. fluorescens, however, does not require acetyl coenzyme A for its activation and it is not affected by the feedback inhibition caused by aspartate (S. Milrad de Forchetti et al., J. Gen. Microbiol., 93, 75-81 (1976)). Overproduced P. fluorescens pyruvate carboxylase should allow even more carbon flow to be diverted towards oxaloacetate.

Because the genes encoding pyruvate carboxylases in bacteria appear to be highly homologous, the P. fluorescens pyc gene may be readily isolated from a genomic library using probes which have been prepared from the R. etli gene. The gene for pyruvate carboxylase in P. fluorescens will thus be identified, isolated, and cloned into an expression vector using standard genetic engineering techniques. Alternatively, the pyruvate carboxylase enzyme can be isolated and purified from P. fluorescens by following pyruvate carboxylase activity (as described in the above Examples) and also by assaying for biotinylated protein using Western blots. The N-terminal amino acid sequence of the purified protein is determined, then a degenerate oligonucleotide probe is made which is used to isolate the gene encoding pyruvate carboxylase from a genomic library that has been prepared from P. fluorescens. The pyc clone thus obtained is sequenced. From the sequence data, oligonucleotide primers are designed that allow cloning of this gene into an expression vector so that pyruvate carboxylase can be overproduced in the host cell. Either method can be used to yield a vector encoding the P. fluorescens pyc gene, which is then used to transform the host E. coli or C. glutamicum cell. Pyruvate carboxylase from P. fluorescens is expressed in the host cell, and biochemical production is enhanced as described in the preceding examples.

### EXAMPLE X

### Enhancement of Biochemical Production by Overexpression of Both Pyruvate Carboxylase and PEP Carboxylase

In many organisms PEP can be carboxylated to oxaloacetate via PEP carboxylase or it can be converted to pyruvate by pyruvate kinase (I. Shiio et al., J. Biochem., 48, 110-120 (1960);M. Jetten et al., Appl. Microbiol. Biotechnol., 41, 47-52 (1994)). One possible strategy that was tried to increase the carbon flux toward oxaloacetate in C. glutamicum was to block the carbon flux from PEP toward pyruvate (see FIG. 3). However, lysine production by pyruvate kinase mutants was 40% lower than by a parent strain, indicating that pyruvate is essential for high-level lysine production (M. Gubler et al., Appl. Microbiol. Biotechnol., 60, 857-863 (1994)).

Carbon flux toward oxaloacetate may be increased by overexpressing PEP carboxylase in conjunction with overexpressed pyruvate carboxylase without concoimtantly blocking carbon flux from PEP to pyruvate or affecting glucose uptake.

In heterotrophs such as C. glutamicum, however, PEP carboxylase requires acetyl-CoA for its activation, and is inhibited by aspartate (M. Jetten et al., Annals NY Acad. Sci., 272, 12-29 (1993)); hence amplification of C. glutamicum PEP carboxylase genes has not resulted in increased lysine yield (J. Cremer et al., Appl. Environ. Microbiol., 57, 1746-1752 (1991)). PEP carboxylase isolated from the cyanobacteria Anacystis nidulans, however, does not require acetyl CoA for activation nor is it inhibited by aspartate (M. Utter et al., Enzymes, 6, 117-135 (1972)). Therefore, this heterologous enzyme can be used to increase the carbon flux towards oxaloacetate in C. glutamicum. The genes encoding PEP carboxylase in A. nidulans have been isolated and cloned (T. Kodaki et al., J. Biochem., 97, 533-539 (1985)).

### EXAMPLE XI

### Enhancement of Biochemical Production by Disrupting the pck Gene Encoding PEP Carboxykinase in Conjunction with Overexpressed Pyruvate Carboxylase

Some of carbon which is diverted to oxaloacetate via overproduced pyruvate carboxylase is likely converted back to PEP due to the presence of PEP carboxykinase. More carbon can be diverted towards oxaloacetate in these systems if the host cell contains a disrupted pck gene, such as an E. coli strain which contains a pck null allele (e.g., A. Goldie, J. Bacteriol., 141, 1115-1121 (1980)).

### EXAMPLE XII

### Pyruvate Carboxylase Increases Anaerobic Fumarate Production in E. coli AFP111

The objective of this study was to determine how pyruvate carboxylase affected the production of the key metabolites succinate, fumarate, pyruvate, acetate, and ethanol in the E. coli strains NZN111 and AFP111 grown under strict anaerobic conditions.

Materials and Methods

Strains and plasmids. All strains and plasmids used in this study are listed in Table 7. The ppc gene encodes for the enzyme PEP carboxylase. To construct AFP111 Δppc, a P1 lysate from ALS804 was used to transduce AFP111 to Tet(R). To verify that the ppc::kan deletion had been introduced into AFP111, a P1 lysate was prepared from AFP111 Δppc and used to transduce MG1655 to Tet(R). The MG1655 Tet(R) transductant colonies were then scored for Kan(R) to show that the ppc::kan deletion was linked to the zii-510::Tn10 transposon as expected. To construct ALS804, a P1 lysate from CGSC6390 was used to transduce JCL1242 to Tet(R) on Rich Tet Kan media in order to preserve the ppc::kan deletion.

Fermentation media. Anaerobic fermentations contained 25 g/L Luria-Bertani (LB) broth and 10 g/L glucose. The pH of the media was maintained between 6.7 and 7.3 by supplementing the media with 40 g/L MgCO₃. All media were supplemented with 1.0 mg/L biotin and 1.0 mg/L thiamine and 100 mg/L ampicillin for the strains that contained the pTrc99A-pyc plasmid. Pyruvate carboxylase expression was induced by the addition of isopropyl-β-D-thiogalactopyranoside (IPTG) to a final concentration of 1.0 mM, unless otherwise indicated.

Growth conditions. Anaerobic fermentations of 100 mL were performed in serum bottles under an atmosphere of pure CO₂ or pure H₂ and agitated at 250 rpm. Serum bottles were inoculated with 10 mL of aerobically grown culture. All fermentations were performed at 37° C. in triplicate from independent inocula. Statistical analyses were completed using Student's t-test, and P<0.10 was considered the criterion for significance.

Analyses. Cell growth during the aerobic phase was monitored by measuring the optical density (OD) at 550 nm (DU-650 UV-Vis spectrophotometer, Beckman Instruments, San Jose, Calif.). Optical density during the anaerobic phases was not measured due to interference by solid MgCO₃. Samples were centrifuged (10,000×g for 10 minutes at 25° C.), and the supernatant analyzed for sugars, organic acids and ethanol by high pressure liquid chromatography as previously described (Eiteman et al., 1997, Anal. Chim. Acta 338:69-75).

Enzyme assays. Cell-free extracts of the E. coli strains were prepared by washing the cell pellet with an appropriate buffer and disrupting the suspended cells using the SLM-Aminco FRENCH pressure cell (Spectronic Instruments, NY) at a pressure of 14,000 psi. Cell debris were removed by centrifugation (20,000×g for 15 minutes at 4° C.), and the cell-free extract used to measure enzyme activities. The following enzymes were examined: acetate kinase, fumarate reductase, glucokinase, isocitrate dehydrogenase, isocitrate lyase, phosphoenolpyruvate carboxylase, pyruvate carboxylase. For all cases, one unit of enzyme activity is the quantity of enzyme that converts 1 μmole of substrate to product per minute. Total protein in the cell-free extract was determined using bovine serum albumin as the standard.

Results

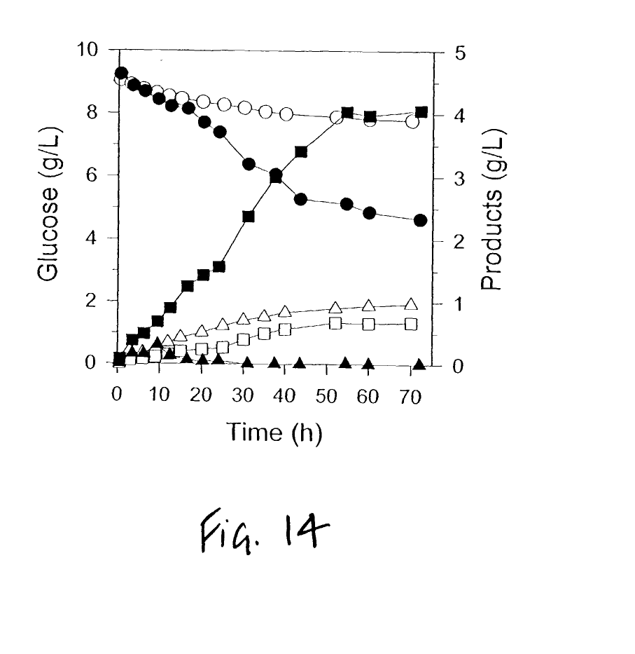

Substrate and products during exclusively anaerobic growth. We first compared the products formed during exclusively anaerobic fermentations of E. coli NZN111 and AFP111 with and without pTrc99A-pyc. Fermentations of NZN111 without pTrc99A-pyc were terminated at 72 hours after the rate of succinate production ceased (FIG. 14). For this strain, glucose was consumed very slowly (0.018 g/Lh), and about 1.0 g/L pyruvate and 0.6 g/L succinate were the principal end products. AFP111 (FIG. 14) consumed glucose more quickly (0.057 g/Lh) and generated succinate to a final concentration of 4.0 g/L. Pyruvate accumulated to about 0.4 g/L at 10 hours, before itself being consumed completely by about 30 hours.

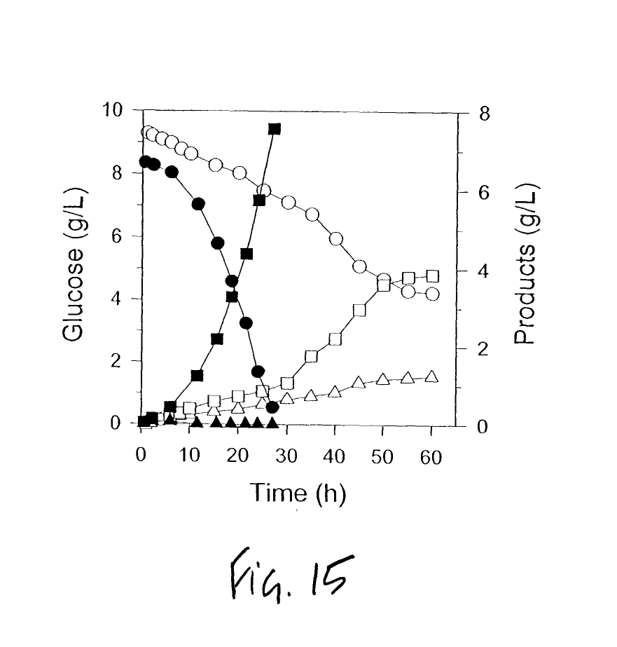

We studied two levels of pyruvate carboxylase expression for both strains: minimal pyruvate carboxylase expression by excluding IPTG and a comparatively high level of pyruvate carboxylase expression using 1.0 mM IPTG. Without IPTG induction NZN111 /pTrc99A-pyc consumed glucose and produced succinate 4-6 times faster than NZN111 (FIG. 15). Also, NZN111/pTrc99A-pyc yielded a final succinate concentration of 4.0 g/L, and a final pyruvate concentration of 1.2 g/L. Without induction AFP111/pTrc99A-pyc similarly consumed glucose and generated succinate more quickly than AFP111, reaching a succinate concentration of nearly 8.0 g/L (FIG. 15).

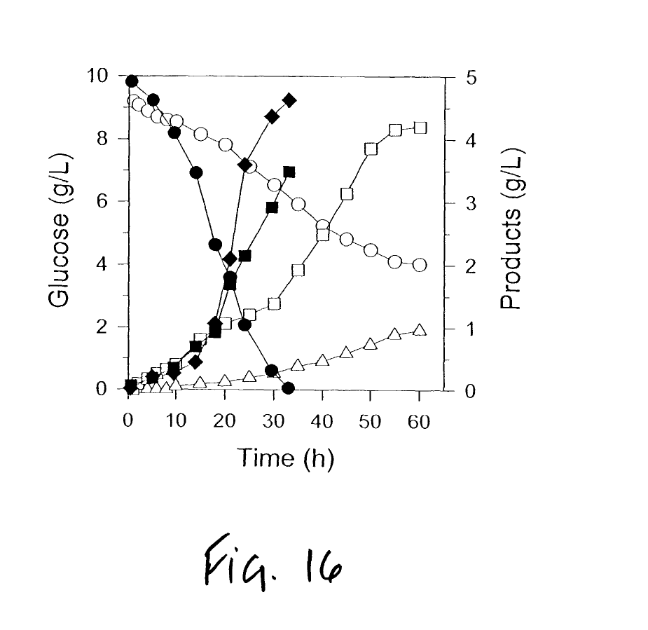

Fermentations using NZN111/pTrc99A-pyc in the presence of 1.0 mM IPTG were similar to fermentations using this strain without IPTG induction (FIG. 16). AFP111/pTrc99A-pyc with IPTG also consumed glucose at the same rate (0.28 g/Lh) as this strain without induction. However, for AFP111/pTrc99A-pyc both succinate and fumarate were significant products with a molar succinate:fumarate ratio of 43:57 and a combined productivity of 0.25 g/Lh. Similar to other AFP111 fermentations, pyruvate accumulated slightly at 10 hours before being consumed. When hydrogen was used in the headspace instead of carbon dioxide for AFP111/pTrc99A-pyc fermentations (with IPTG), fumarate did not accumulate, and the succinate productivity was 0.35 g/Lh.

Product yields in exclusively anaerobic fermentations are summarized in Table 8. For NZN111 strains, increasing the level of pyruvate carboxylase expression resulted in increased succinate and reduced pyruvate accumulation. For AFP111 strains, a low level of pyruvate carboxylase expression resulted in an insignificant increase in succinate compared to when the pyc gene was absent. However, a high level of pyruvate carboxylase expression resulted in both succinate and fumarate generation. Replacement of carbon dioxide in the headspace with hydrogen restored the succinate yield.

Enzyme activities during exclusively anaerobic growth. We also compared the enzyme activities during exclusively anaerobic fermentations of NZN111 and AFP111 with and without pTrc99A-pyc. Specific activities were measured for seven enzymes involved in the formation of the products (Table 9). In NZN111 and AFP111, PEP carboxylase is the only enzyme that directs carbon towards oxaloacetate (OAA) for succinate production. When grown under exclusively anaerobic conditions, several significant differences in specific enzyme activities were observed between NZN111 and AFP111. AFP111 showed much greater activities than NZN111 for acetate kinase (about 5 times greater), fumarate reductase (twice as great) and glucokinase (about 50 times greater). AFP111 was also observed to have slightly greater activity of PEP carboxylase.

As expected, full induction of the pyc gene with 1.0 mM IPTG resulted in the greatest pyruvate carboxylase activities for both NZN111/pTrc99A-pyc and AFP111/pTrc99A-pyc. Lower but significant activities were observed in these strains without IPTG addition. The activities of acetate kinase, fumarate reductase and glucokinase generally increased with increasing pyruvate carboxylase activity for both strains. In contrast, the activity of PEP carboxylase decreased with increasing pyruvate carboxylase activity. Indeed, the sum of PEP carboxylase and pyruvate carboxylase activities (about 0.11 U/mg protein) was not significantly different for NZN111, NZN111/pTrc99A-pyc without IPTG and NZN111/pTrc99A-pyc with IPTG. Except for those cases using hydrogen in the headspace, the sum of the activities of these two enzymes was 0. 13-0.17 for AFP111, AFP111/pTrc99A-pyc without IPTG and AFP111/pTrc99A-pyc with IPTG. Activities for isocitrate lyase and isocitrate dehydrogenase were not detected during exclusively anaerobic fermentations for any of the strains. Using hydrogen in the headspace instead of carbon dioxide for AFP111/pTrc99A-pyc resulted in the greatest enzyme activities observed during anaerobic growth for acetate kinase, glucokinase and pyruvate carboxylase.

TABLE 7
Strains and plasmids used.

| Strain/plasmid | Relevant characteristics | Reference |
|---|---|---|
| NZN111 | F⁺ λ⁻ rpoS396(Am) rph-1_ | Bunch et al., |
| | (pflAB::Cam) ldhA::Kan | Microbiol. 143:187- |
| | | 195, 1997 |
| AFP111 | NZNIII ptsG | Donnelly et al., |
| | | Appl. Biochem. |
| | | Biotechnol. |
| | | 70-72:187-198, |
| | | 1998; Chatterjee |
| | | et al., Appl. |
| | | Environ. Microbiol. |
| | | 67:148-154, 2001. |
| CGSC6390 | thr-1 araC14 leuB6 fhuA31 lacY1 | E. coli Genetic |
| | tsx-78 Δ[galK-att(λ)]99 λ⁻ eda-50 | Stock |
| | hisG4(Oc) rpsL136(strR) xylA5 | Center |
| | mtl-1 zii-510::Tn10 metF159(Am) | |
| | thi-1 | |
| MG1655 | wild type (F⁻λ⁻) | M. Guyer et al., |
| | | Quant. |
| | | Biol., Cold Spring |
| | | Harbor Symp., 45, |
| | | 135-140 (1981) |
| JCL1242 | F⁻λ⁻ Δ(argF-lac)U169ppc::Kan | P. Chao et al., Appl. |
| | | Env. Microbiol., 59, |
| | | 4261-4265 (1993) |
| ALS804 | JCL1242 zii-510::Tn10 | This example |
| AFP111 Δppc | AFP111 ppc::Kan | This example |
| pTrc99A-pyc | R. etli pyc bla lacl^{q} trc ColE1 | Example II |

TABLE 8
Mass yields of products during exclusively anaerobic growth on glucose-rich media. Serum bottles
were under an atmosphere of CO₂ or H₂.

| | | | Yield (g product/g glucose) | | | | |
|---|---|---|---|---|---|---|---|
| Strain | Headspace | IPTG (mM) | succinate | pyruvate | acetate | ethanol | fumarate |
| NZN111 | CO₂ | 0.0 | 0.53 a | 0.76 a | 0.06 a | 0.06 a | 0.00 a |
| NZN111/pTrc99A-pyc | CO₂ | 0.0 | 0.77 b | 0.25 b | 0.09 b | 0.03 b | 0.00 a |
| NZN111/pTrc99A-pyc | CO₂ | 1.0 | 0.81 c | 0.19 c | 0.11 c | 0.05 c | 0.00 a |
| AFP111 | CO₂ | 0.0 | 0.88 d | 0.00 d | 0.22 d | 0.07 d | 0.00 a |
| AFP111/pTrc99A-pyc | CO₂ | 0.0 | 0.96 d | 0.00 d | 0.23 c | 0.06 ade | 0.07 b |
| AFP111/pTrc99A-pyc | CO₂ | 1.0 | 0.35 e | 0.00 c | 0.09 abc | 0.06 ae | 0.47 c |
| AFP111/pTrc99A-pyc | H₂ | 1.0 | 0.91 c | 0.00 d | 0.11 c | 0.07 ade | 0.00 a |

†Yields followed by differing letters are significantly different at 90% confidence level.

TABLE 9
Enzyme activities during exclusively anaerobic growth on glucose-rich media. Serum bottles were
under an atmosphere of CO₂ or H₂.

| | | IPTG | Specific Activity (U/mg protein)† | | | | | | |
|---|---|---|---|---|---|---|---|---|---|
| Strain | Headspace | (mM) | ACK | FR | GK | ICDH | ICL | PPC | PYC |
| NZN111 | CO₂ | 0.0 | 0.20 ab | 0.17 a | 0.018 a | 0.00 | 0.00 | 0.10 a | 0.00 a |
| NZN111/pTrc99A-pyc | CO₂ | 0.0 | 0.15 a | 0.26 b | 0.025 a | 0.00 | 0.00 | 0.061 b | 0.050 b |
| NZN111/pTrc99A-pyc | CO₂ | 1.0 | 0.27 b | 0.33 c | 0.11 b | 0.00 | 0.00 | 0.020 c | 0.086 bc |
| AFP111 | CO₂ | 0.0 | 1.11 c | 0.45 cd | 0.98 c | 0.00 | 0.00 | 0.13 d | 0.00 ad |
| AFP111/pTrc99A-pyc | CO₂ | 0.0 | 1.24 c | 0.52 d | 1.08 d | 0.00 | 0.00 | 0.11 ad | 0.056 bd |
| AFP111/pTrc99A-pyc | CO₂ | 1.0 | 1.42 d | 0.68 e | 1.30 e | 0.00 | 0.00 | 0.057 b | 0.12 c |
| AFP111/pTrc99A-pyc | H₂ | 1.0 | 1.79 e | 0.74 e | 1.58 f | 0.00 | 0.00 | 0.099 a | 0.17 e |

†Enzyme activities followed by differing letters show statistically significant difference at 90% confidence level.
Enzyme abbreviations: ACK, acetate kinase; FR, fumarate reductase; GK, glucokinase; ICDH, isocitrate dehydrogenase; ICL, isocitrate lyase; PPC, PEP carboxylase; PYC, pyruvate carboxylase.

Discussion

In this study we compared two doubly mutated (ldh pfl) strains of E. coli, NZN111 and AFP111, in the absence and presence of the enzyme pyruvate carboxylase using anaerobic growth conditions. The synthesis of oxaloacetate (OAA) is a key step in the production of succinate. In most eukaryotes and some prokaryotes OAA is replenished both from PEP and from pyruvate by PEP carboxylase and pyruvate carboxylase, respectively. However, in wild-type E. coli PEP carboxylase is the principal anaplerotic reaction to replenish OAA. That portion of PEP not flowing to OAA is converted to pyruvate, and under anaerobic conditions for NZN111 and AFP111 (in the absence of the assimilating enzymes lactate dehydrogenase and pyruvate formate lyase) pyruvate was observed to accumulate. By transforming these strains with pTrc99A-pyc which expresses pyruvate carboxylase from R. etli, E. coli is provided with another anaplerotic route to OAA formation (Example I), and the strains show reduced pyruvate accumulation and concomitant increased succinate production. Although all these strains grew very slowly, we observed increased cell growth rates and glucose consumption rates for either of these strains when this additional anaplerotic route was available.

NZN111 and AFP111 are different. NZN111 has been reported to grow very slowly on glucose in the absence of oxygen while AFP111 isolated as a result of a ptsG mutation in NZN111 grows more quickly. Both strains have been reported to accumulate significant quantities of succinate during anaerobic growth (Stols et al., 1997, Appl. Biochem. Biotechnol. 63-65:153-158; Stols et al., 1997, Appl. Environ. Microbiol. 63:2695-2701; Nghiem et al. U.S. Pat. No. 5,869,301). The significant findings in this study are the demonstration of enhanced glucokinase activity in AFP111 strains, and the observation of no isocitrate lyase activity when either strain is grown anaerobically. (Isocitrate lyase activity was observed after aerobic growth.)

Our results show two means of glucose consumption and two paths from PEP to succinate. The two general routes which E. coli uses to transport and phosphorylate glucose differ in E. coli strains NZN111 and AFP111. One route involves two multienzyme systems collectively termed the phosphotransferase system (PTS) which concomitantly transport and phosphorylate glucose to intracellular glucose 6-phosphate by using PEP as a cosubstrate. This route ultimately leads to the formation of both PEP and pyruvate, and the resulting net reaction may be expressed as:

glucose→pyruvate+PEP+4 H+2 H₂O+ATP

The one mole of PEP formed in this reaction is available to PEP carboxylase to generate OAA, or to pyruvate kinase to generate a second mole of pyruvate and ATP. The one mole committed to pyruvate is not available for direct conversion to OAA. Wild-type E. coli can still grow in the absence of the PTS, and a mutation in the glk gene for glucokinase is necessary to eliminate growth on glucose completely. Thus, a second route for glucose uptake involves glucose transport uncoupled from phosphorylation, a route which generally appears to be insignificant compared to the PTS in wild-type E. coli. The resulting net reaction may be expressed as:

glucose→PEP+4 H+2 H₂O+0 ATP

In this case, two moles of PEP are available to PEP carboxylase for OAA formation. Of course, one mole of PEP could form pyruvate via pyruvate kinase with the generation of ATP so that the ultimate equations for the two routes to pyruvate are equivalent. In this study for anerobically grown cells, AFP111 showed markedly greater glucokinase activity than NZN111.

From three carbon intermediates, succinate may be formed by two means: via the reductive arm of the TCA cycle, or via the glyoxylate shunt. The reductive branch of the TCA cycle converts OAA into malate, fumarate and then succinate. From a three carbon precursor of OAA (PEP or pyruvate), this path requires the incorporation of four electrons and one mole of CO₂. The net equation of this C3+C1 pathway is:

PEP+CO₂+4 H→succinate+0 ATP

The glyoxylate shunt operates as a cycle to convert two moles of acetyl CoA into succinate. From two moles of the three carbon precursor pyruvate, one cycle around the glyoxylate pathway generates six electrons and two moles of CO₂. The net equation of this C2+C2 pathway is:

2 pyruvate→succinate+6 H+2 CO₂+0 ATP

The glyoxylate shunt has not previously been shown to be important in the formation of succinate, and it is most commonly associated with microbial growth on acetate. In this study, the key glyoxylate shunt enzyme isocitrate lyase was not detected with either strain grown under anaerobic conditions, but was detected after aerobic growth. Because the two strains differ in their mode of glucose uptake, and growth conditions affect the expression of isocitrate lyase, one would expect differences in the distribution of end-products between the two strains and during anaerobic and aerobic growth.

Both glucose consumption routes to three carbon intermediates generate 4 electrons per glucose. Since the C3+C1 pathway requires 8 electrons per mole glucose to form 2 moles of succinate, and the C2+C2 pathway generates 6 electrons to form one mole of succinate per mole glucose, neither of these two succinate-producing pathways alone is sufficient to balance the electrons in the overall conversion of glucose to succinate. The maximum possible succinate yield to achieve a redox balance is 1.714 moles succinate from one mole of glucose, providing a mass yield of 1.12. In the absence of an additional electron donor, this maximum theoretical yield necessitates both pathways function from 3-carbon intermediates to succinate and that specifically 71.4% of the carbon flow to OAA and 28.6% of the carbon flow to acetyl CoA. If the glyoxylate shunt is not active, as we observed during exclusively anaerobic growth, then this maximal yield of succinate can not be achieved.

Without the pyc gene NZN111 and AFP111 have only one means for PEP to flow directly to OAA, and that is via PEP carboxylase. For these strains, the two routes for glucose uptake result in vastly different maximal succinate yields. For a strain relying on the PTS for glucose uptake (NZN111), because half of the carbon is committed to pyruvate by the PTS, only 50% of the carbon is available for subsequent conversion to succinate via the C3+C1 pathway. This fraction is lower than the 71.4% needed for a maximum theoretical yield of 1.12. The maximum succinate yield is in this case attained when the one mole of PEP generated from glucose is entirely converted to OAA. Such a scenario satisfying a redox balance could generate 1.20 moles succinate per mole of glucose with 17% of the succinate coming from the glyoxylate shunt for a mass yield of 0.79. For a strain relying on glucokinase for glucose uptake (AFP111), all carbon from glucose is available for subsequent conversion to succinate via the C3+C1 pathway. In this case, 28.6% of the PEP could flow through pyruvate kinase to pyruvate to achieve the maximum succinate mass yield of 1.12 satisfying a redox balance.

The differences between the observed activities of glucokinase in NZN111 and AFP111 demonstrate a difference in the flexibility of each organism. During anaerobic growth of NZN111 with the PTS dominating glucose uptake, nearly one-half of the carbon is committed to pyruvate. In the absence of isocitrate lyase activity, we observed pyruvate to accumulate to about twice the final molar concentration of succinate (Table 8). During anaerobic growth of AFP111, with glucose uptake occurring via glucokinase, less glucose is committed to pyruvate. All carbon could therefore potentially be diverted to succinate via OAA, and we observed no pyruvate at the end of AFP111 fermentations.

The level of pyruvate carboxylase activity affects the final product distribution with fumarate the redox-balanced end-product. As noted above, two moles of NADH (4H) are produced for every mole of glucose consumed during glycolysis. NADH must be reoxidized to NAD for the fermentation to progress. This reoxidation is achieved by the reduction of OAA to either fumarate, which requires one mole of NADH, or succinate, which requires two moles of NADH. If all the carbon from PEP were to flow to OAA, we would expect fumarate to be the exclusive end-product which balances the NADH generated in glycolysis. In fact, if greater than 71.8% of the carbon from PEP were to flow to OAA, a redox balance necessitates fumarate to be present in addition to succinate. Thus, in those cases where both pyruvate carboxylase and PEP carboxylase activities are high and activities of other pyruvate assimilating enzymes such as isocitrate lyase are low, the large fraction of PEP expected to flow to OAA would result in some fumarate accumulation. For AFP111/pTrc99A-pyc grown anaerobically with IPTG (with no isocitrate lyase activity and hence limited pyruvate assimilation), we indeed did observe fumarate to accumulate to a molar fumarate to succinate ratio of 1.33. Growing AFP111/pTrc99A-pyc anaerobically in the presence of hydrogen in contrast prevented the accumulation of fumarate, suggesting that the strains have a mechanism for regenerating NAD using hydrogen. Both pyruvate carboxylase and isocitrate lyase activities are needed for optimal succinate production. High pyruvate carboxylase activity and the absence of isocitrate lyase activity is needed for fumarate production.

Another important result is that the presence of pyruvate carboxylase via the pTrc99A-pyc plasmid increased the rates of both glucose consumption and cell growth. This result is contrary to common observations that the expression of heterologous cloned genes substantially reduces cell growth rate (Diaz Ricci et al., 2000, Crit. Rev. Biotechnol. 20(2):79-108). Furthermore, increases in glucose uptake rate have been proposed to be due to enhanced expression of proteins involved in the PTS (Diaz Ricci et al., 1995, J. Bacteriol. 177:6684-6687). In the current study, AFP111 with pyruvate carboxylase averaged five times greater glucose uptake rate under anaerobic conditions and achieved a 50% greater cell density after 8 hours under aerobic conditions as compared to AFP111 without pyruvate carboxylase. Since this organism appears not to have a significant PTS for glucose uptake, additional studies are needed to reconcile the reason that the growth and glucose uptake of this particular multi-mutated strain benefits from the additional anaplerotic reaction afforded by pyruvate carboxylase.

In summary, the glyoxylate shunt is a key pathway for the accumulation of succinate and fumarate by the two pfl ldh strains of E. coli we studied. Active during aerobic growth in these strains, the glyoxylate shunt provides a means for these organisms to sustain a redox balance under subsequent anaerobic conditions and generate succinate. However, under anaerobic growth the absence of isocitrate lyase activity (by virtue of the growth conditions), forces fumarate accumulation in a strain with high pyruvate carboxylase activity. If pyruvate carboxylase activity is absent, then a large fraction of the carbon becomes the dead-end product pyruvate. These results suggest that fumarate accumulation could be further increased using genetic or operational steps which either altogether remove isocitrate lyase activity (for example during aerobic growth prior to an anaerobic production phase) or which additionally increase the activity of pyruvate carboxylase.

### EXAMPLE XIII

### Succinate Production in Dual-Phase E. coli Fermentations

E. coli AFP111 is a pfl ldhA strain that can grow anaerobically on glucose as the sole carbon source. This strain was has a mutation in the ptsG gene which encodes for an enzyme of the phosphotransferase system (PTS). Because of the ptsG mutation, AFP111 relies on glucokinase for glucose uptake. When grown aerobically for biomass generation and then subject to anaerobic conditions (a “dual-phase” fermentation), AFP111 attains succinate yields and productivities of 0.99 and 0.87 g/Lh, respectively (P. Nghiem et al. U.S. Pat. No. 5,869,301).

Our objective was to study how the time of transition from aerobic to anaerobic phases and the presence of pyruvate carboxylase activity affects succinate production in dual-phase fermentations by E. coli AFP111.

Materials and Methods

Strains and plasmids. E. coli AFP111 (F⁺ λ⁻ rpoS396(Am) rph-1 ΔpflAB::Cam ldhA::Kan ptsG) was the only strain used in this study (M. Akesson et al., Biotechnol. Bioeng. 64:590-598 (1999); P. Nghiem et al. U.S. Pat. No. 5,869,301). AFP111 was transformed with the pyc gene from Rhizobium etli using the pTrc99A-pyc plasmid (R. etli pyc Ap^{R} trcPO lacI^{q} ColE1 ori) as described previously (R. Gokarn et al., Appl. Microbiol. Biotechnol. 56:188-195 (2001)).

Fermentation media. All fermentations used complex media containing (g/L): glucose, 40; yeast extract, 10; tryptone, 20; K₂HPO₄.3H2O, 0.90; KH₂PO₄, 1.14; (NH₄)₂SO₄, 3.0; MgSO₄.7H₂O, 0.30 and CaCl₂.2H₂O, 0.25. The media was supplemented with 1.0 mg/L biotin and 1.0 mg/L thiamine. For the pyc-containing strains the media also contained 100 mg/L ampicillin. Since significant pyruvate carboxylase activity exists without the addition of a chemical inducer (see Example XIII), most studies were performed without inducer. For a final optimized fermentation, pyruvate carboxylase expression was induced to a greater level by the addition of isopropyl-β-D-thiogalactopyranoside (IPTG) to a final concentration of 1.0 mM.

Growth conditions. The 37° C. fermentations had an initial volume of 1.5 L in 2.5 L Bioflow II fermenters (New Brunswick Scientific Instruments, New Brunswick, N.J.). Inocula of 100 ml used the same media as the fermenter and were grown in shake flasks for 6 hours at 37° C. A series of exclusively aerobic fermentations (i.e., without a transition to anaerobic conditions) were first completed in order to catalog the changes in the physiological states of AFP111 and AFP111/pTrc99A-pyc during the course of the aerobic growth phase. Constant agitation rates of 500 rpm and 750 rpm were studied, corresponding to volumetric oxygen mass transfer coefficients (k_{L}a) of 52 h⁻¹ and 69 h⁻¹ respectively, as calculated by the method of Taguchi and Humphrey (J. Ferm. Technol. 44:881-889 (1966)). The air flow rate was maintained at 1.20 L/min by mass flow controllers (Unit Instruments Inc., Orange, Calif.). The pH was controlled at 7.0 with 20% NaOH and 20% HCl. The dissolved oxygen concentration (DO) was monitored with an on-line probe (Mettler-Toledo Process Analytical Instruments, Wilmington, Mass.). The oxygen and CO₂ concentrations in the off-gas were measured by a gas analyzer (Ultramat 23, Siemens AG, Munich, Germany) and used to calculate the respiratory quotient (RQ).

The activities of several key enzymes of the central metabolism were also measured at regular intervals during aerobic growth: glucokinase, PEP carboxylase, pyruvate carboxylase, pyruvate dehydrogenase, isocitrate lyase and fumarate reductase. Cell-free extracts were prepared by washing the cell pellet with an appropriate buffer and disrupting the suspended cells using the FRENCH pressure cell (ThermoSpectronic, Rochester, N.Y.) at a pressure of 14,000 psi. Cell debris were removed by centrifugation (20,000×g for 15 min at 4° C.), and the cell-free extract was used for measuring the enzyme activities. For all cases, one unit of enzyme activity is the quantity of enzyme that converts 1 μmol of substrate to product per minute at the optimum pH and temperature. Total protein in the cell-free extract was determined using bovine serum albumin as the standard. Based on milestones observed in the course of these aerobic fermentations, several transition times were selected for further study.

Dual-phase fermentations were initiated as described for the aerobic fermentations. At each selected transition time, oxygen-free CO₂ was sparged at 0.2 L/min to replace air, and the agitation was reduced to 250 rpm. The pH was allowed to drift to 6.8, at which point it was controlled with 2.0 M Na₂CO₃. Glucose concentration was permitted to decrease to 3 g/L and then maintained at this level with an on-line analyzer (2700 Select, YSI Inc., Yellow Springs, Ohio) by the controlled addition of a sterile 500 g/L glucose feed solution.

Analyses. Cell growth was monitored by measuring the optical density (OD) at 550 nm (DU-650 UV-Vis spectrophotometer, Beckman Instruments, San Jose, Calif.) and correlating with Dry Cell Weight (DCW). Samples were centrifuged (10,000×g for 10 min at 25° C.), and the supernatant analyzed for glucose and all products by high-pressure liquid chromatography (HPLC).

Results

Physiological parameters during aerobic growth in the absence of pyruvate carboxylase. We first conducted exclusively aerobic fermentations using AFP111 in order to find distinguishable growth stages, and thereby define physiological “milestones” which could be used to transition to an anaerobic production phase. We compared these fermentations at k_{L}a values of 52 h⁻¹ (medium transfer rate) and 69 h⁻¹ (high transfer rate). All fermentations were repeated 3-6 times, and consistent results were obtained with respect to the stages observed, though a particular stage generally did not commence at one clock time. Representative fermentations are shown in the figures.

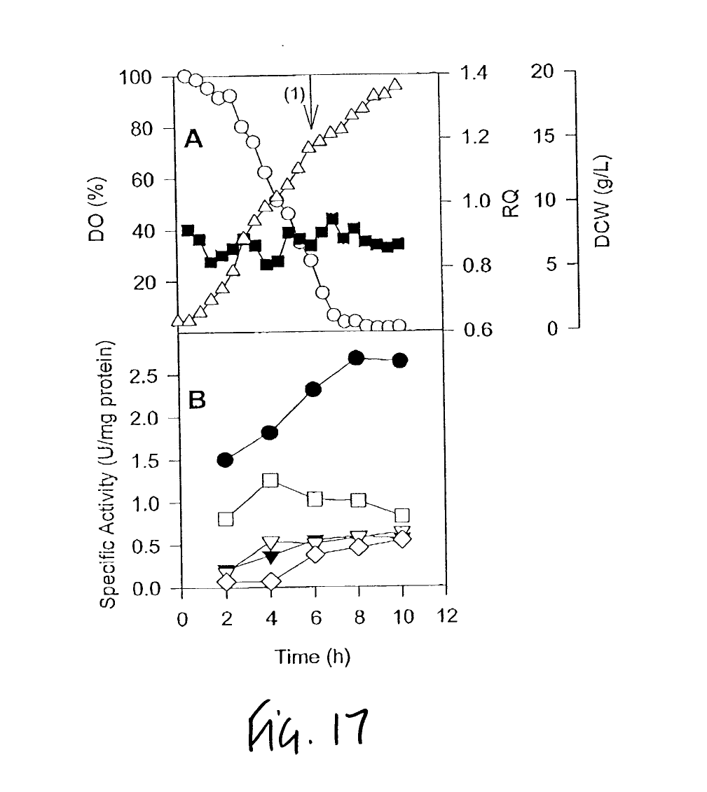

Cell growth of AFP111 for medium transfer rate consistently exhibited three distinct stages (FIG. 17A). Stage I corresponded with exponential growth (μ=0.7-0.8 h⁻¹), high DO and little acetate accumulation. Stage II corresponded with linear growth at 2 g/Lh, decreasing DO and acetate accumulation at over 1 g/Lh. Stage III corresponded with linear growth at 1.0-1.5 g/Lh, oxygen limitation and less than 1 g/Lh acetate accumulation. The specific enzyme activities of pyruvate dehydrogenase and isocitrate lyase increased substantially between stages I and II (FIG. 17B). Also, fumarate reductase activity was very low until just prior to the onset of the third stage. Because the intracellular levels of inhibitors and activators are not known, these in vitro enzyme activities indicate level of active enzyme present but they do not necessarily indicate carbon flowing through a particular pathway.

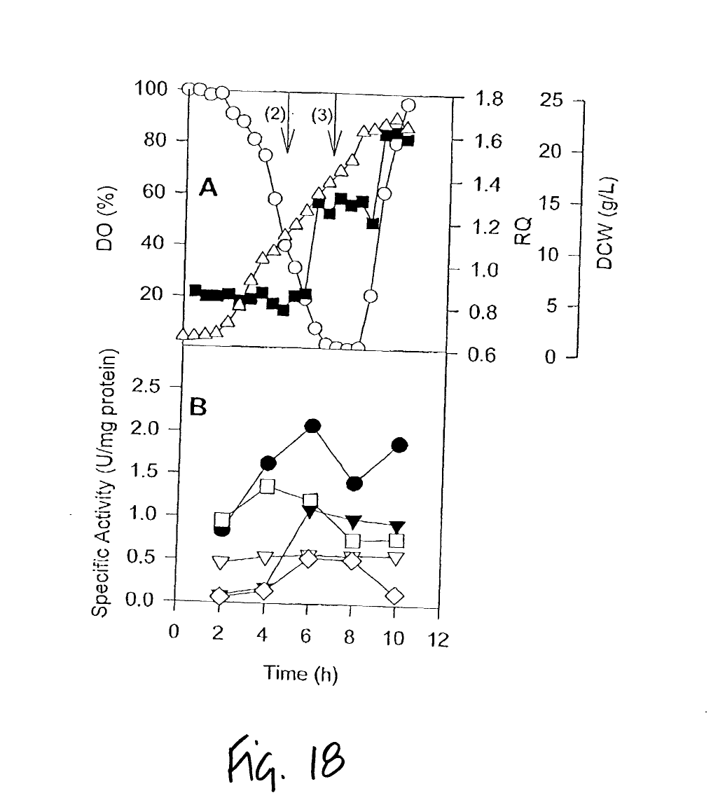

For the fermentation of AFP111 at the high transfer rate (k_{L}a=69 h⁻¹) the fermentations again followed three distinct stages (FIG. 18A). Stage I was an exponential growth phase, and little acetate accumulation. Stage II was again marked by linear cell growth, but in contrast to results at medium transfer rate, acetate did not accumulate during this stage. Also, the RQ abruptly shifted from 0.8-0.9 to 1.2-1.3 when the DO reached about 10%, marking the start of a third stage. During stage III the cell growth rate remained at about 2.0 g/Lh, the DO remained below 10%, the acetate concentration was negligible, and the RQ remained at 1.2-1.3.

In general, the enzyme activities measured during the first 5-6 hours were identical to those observed in the AFP111 fermentations at medium oxygen transfer rate (FIG. 18B). However, between stages II and III the activities of pyruvate dehydrogenase and fumarate reductase increased significantly. The activity of pyruvate dehydrogenase for these fermentations during the third stage (1.1 U/mg) was about twice that observed for the medium transfer rate fermentations, and over eight times greater than observed during stages I and II in the same fermentation. Also, PEP carboxylase activity decreased by over 30% from stage II to stage III. Because the pyruvate dehydrogenase complex generates carbon dioxide while PEP carboxylase consumes carbon dioxide, the increased RQ observed during phase III may be a consequence of a net increase in carbon dioxide generated by the change in activity of these two enzymes.

Based on these aerobic fermentations of AFP111, we identified three different milestones which could be used to mark a transition between growth and production phases. These times were selected because they were readily distinguishable and broadly represented the observed growth and enzyme activities. The first transition time studied (1) was at conditions of medium transfer rate as the fermentations entered stage III and the DO reached about 10-20% (indicated in FIG. 17A). The second physiological time (2) was at conditions of high transfer rate with the fermentation in stage II (RQ still low), while the third time (3) was taken to be about 1.0 hour after the initiation of stage III when RQ shifted (FIG. 18A).

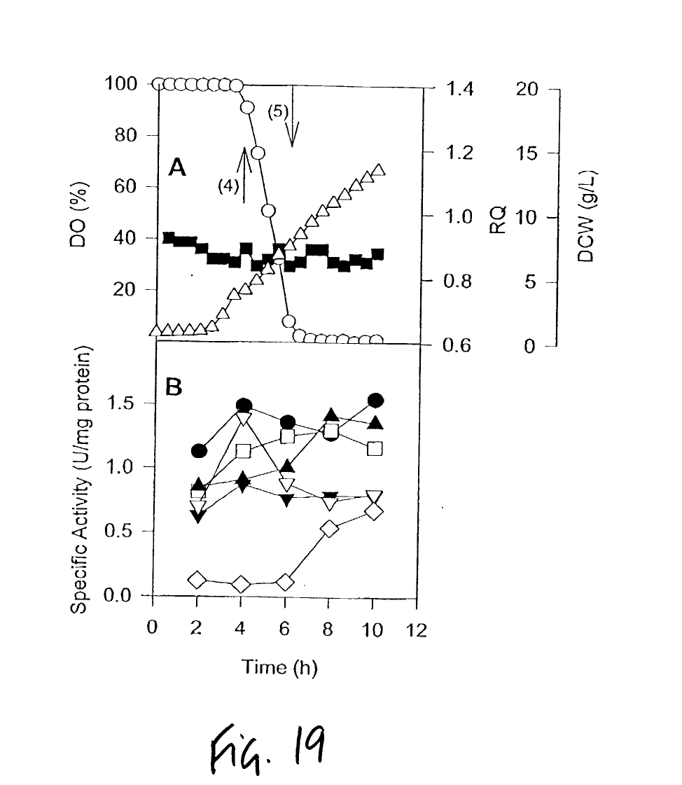

Physiological parameters during aerobic growth in the presence of pyruvate carboxylase. We similarly completed aerobic fermentations of AFP111 with pyruvate carboxylase activity at the two different values of k_{L}a (52 h⁻¹ and 69 h⁻¹). For AFP111/pTrc99A-pyc at medium transfer rate, the DO consistently remained at 90-100% for about 5 hours compared to the 2-3 hours that had been observed for AFP111 at the same transfer rate (FIG. 19A). However, because of the lower cell growth rate for AFP111/pTrc99A-pyc, in both cases the decrease in DO commenced when the cell concentration was about 5 g/L. We observed two distinct stages in these AFP111/pTrc99A-pyc fermentations. The first stage corresponded to high DO and exponential cell growth. The second stage commenced when the DO decreased substantially, and was marked by linear cell growth at about 1.5 g/Lh. The specific activity of fumarate reductase increased after 6 h, but the other enzymatic activities did not appear to follow any trend (FIG. 19B). Furthermore, throughout the fermentation the activity of glucokinase was substantially lower for AFP111/pTrc99A-pyc than we observed for AFP111, while the activities for all the other enzymes were greater for AFP111/pTrc99A-pyc than we observed for AFP111.

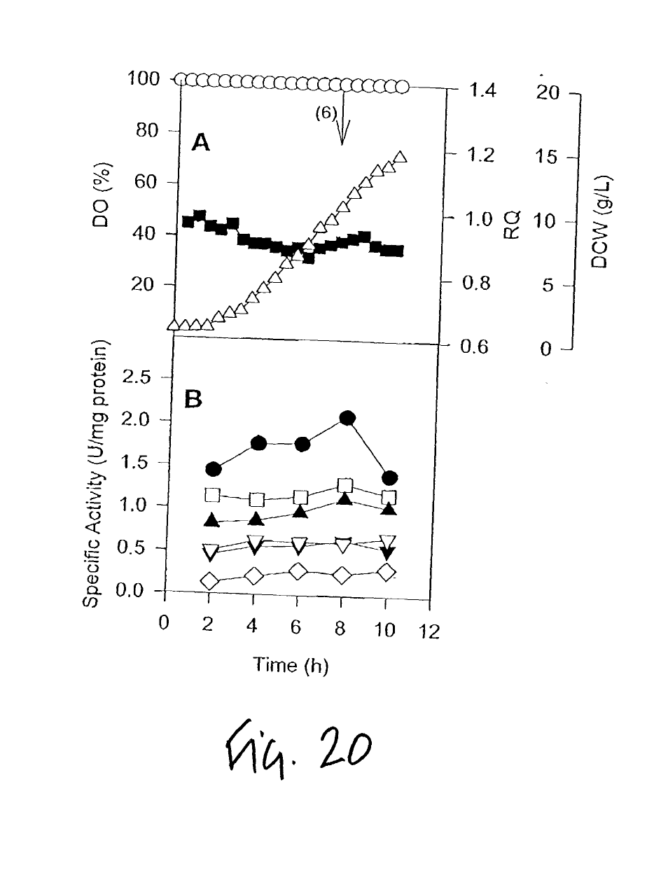

Fermentations of AFP111/pTrc99A-pyc at high transfer rate were markedly different than those fermentations for AFP111 (FIG. 20A). Specifically, the DO concentration remained at 100% and the RQ at 0.8-0.95 throughout the entire course of the fermentations, and cell growth proceeded at a constant rate. The enzyme activities (FIG. 20B) also did not indicate any dramatic shift during the course of the fermentation. It is interesting to compare the DO for the aerobic fermentations at the time that the cell concentration had reached about 12 g/L. The DO at this cell density was consistently about 40% for AFP111 with a medium transfer rate, similar for AFP111 with a high transfer rate, essentially 0% for AFP111/pTrc99A-pyc with the medium transfer rate, but was invariably 100% for AFP111/pTrc99A-pyc at the high transfer rate.

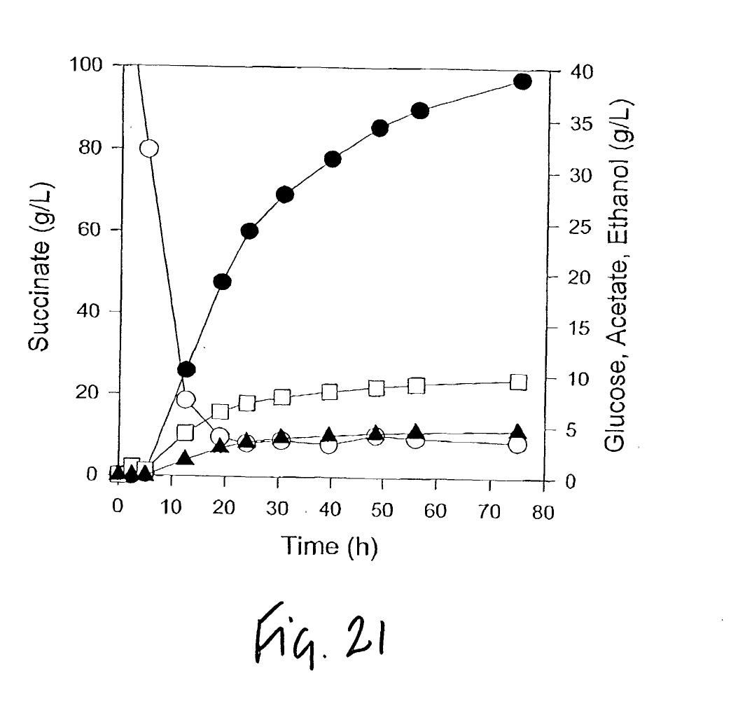

Based on these results, we identified three different physiological milestones during AFP111/pTrc99A-pyc fermentations. The first transition time (4) was at conditions of medium transfer rate the DO began to decrease (indicated in FIG. 21A). The second physiological time (5) was also at conditions of medium transfer rate with the fermentation strongly oxygen limited (DO less than 10%). Because there was no clear distinguishing physiological state during the fermentations of AFP111/pTrc99A-pyc at high transfer rate, the time of transition (6) was arbitrarily selected to be at 8.0 hours, the only one of the six milestones based on a clock time rather than a distinguishable physiological event. Table 10 summarizes the six milestones examined, three transition times for AFP111 and three transition times for AFP111/pTrc99A-pyc.

Dual-phase fermentations. We next studied dual-phase fermentations which included a transition to an anaerobic production phase at each of the six milestones selected from exclusively aerobic fermentations. These fed-batch fermentations were routinely terminated after 48 hours. AFP111 fermentations used milestones #1-3, while AFP111/pTrc99A-pyc fermentations used milestones #4-#6 (see Table 10).

The results of these dual-phase fermentations are summarized in Table 11. The succinate yield was calculated as the mass of product formed in the anaerobic phase divided by the mass of glucose consumed in anaerobic phase. The specific succinate productivity during the anaerobic phase was calculated on the basis of cell concentration at the moment of transition. Generally, total cell mass in the fermenter (taking into account the dilution volume by the glucose feed) decreased by about 10% for AFP111 during the 40 hours anaerobic production phase. In all cases for AFP111/pTrc99A-pyc, however, the total cell mass increased slightly (5-10%) during the course of the anaerobic phase. Fermentations using milestones #1, #4, and #5 resulted in significantly greater volumetric productivities than the other three fermentations.

Thus, both AFP111 and AFP111/pTrc99A-pyc showed greater succinate productivity in an anaerobic phase when the preceding aerobic phase occurred at the medium oxygen transfer rate than when the aerobic growth occurred at the high oxygen transfer rate. Since AFP111/pTrc99A-pyc grew more slowly than AFP111 under the conditions studied, the specific rate of succinate production was the greatest (118 mg/gh) for the fermentation with AFP111/pTrc99A-pyc and milestone #4. The yield of succinate was generally much greater for fermentations using AFP111/pTrc99A-pyc than AFP111.

As milestone #4 appears to be the most promising for succinate production of the six studied, we conducted an extended fed-batch fermentation with AFP111/pTrc99A-pyc (FIG. 21). The final succinate concentration was 97.5 g/L (99.2 g/L succinic acid). The volume of the fermentation increased from 1.5 L to 2.5 L as a result of glucose feed and base addition. The succinate mass yield based on glucose consumed during the anaerobic phase alone was 117%. The overall succinate yield was 110%, and the overall volumetric succinate productivity was 1.3 g/Lh. The final mass ratio of succinate to acetate was 10.2, and the final mass ratio of succinate to ethanol was 21. The cell mass concentration was 10.2 g/L at the transition between growth and production phases. Based on this cell concentration, the specific succinate productivity for the anaerobic phase was 135 mg/gh. Accounting for the dilution volume, cells continued to grow throughout the anaerobic production phase, increasing in mass by 27%.

Discussion

We report here that physiological changes during aerobic growth of two engineered strains of E. coli, AFP111 and AFP111/pTrc99A-pyc, significantly affect succinate production in a subsequent production phase. Different aerobic operational conditions, such as oxygen transfer rates (k_{L}a), would generally be expected to result in different levels of enzyme activity. Moreover, physiological states of an organism can change during the course of aerobic growth as the growth environment changes (for example, through oxygen limitation or product accumulation). These states often become evident in readily measurable parameters such as RQ, DO, component generation and utilization rates, and enzyme activities. The optimal transition time between the two fermentation phases appears to depend on the complex interplay of the activities of numerous enzymes in the two pathways central to succinate production.

With exclusively aerobic fermentations of AFP111 at the high transfer rate we consistently observed an abrupt shift in RQ with a simultaneous increase in the specific activity of pyruvate dehydrogenase. Also, AFP111 at the high transfer rate was never observed to accumulate acetate, while AFP111 at the medium transfer rate (and generally lower pyruvate dehydrogenase activity) did accumulate significant acetate. It is widely believed that when the TCA cycle cannot keep pace with glycolysis, acetate accumulates, a phenomenon known as overflow metabolism (M. Akesson et al., Biotechnol. Bioeng. 64:590-598 (1999); M. Akesson et al., Biotechnol. Bioeng. 73:223-230 (2001); K. Han et al, Biotechnol. Bioeng. 39:663-671 (1992); K. Konstantinov et al., Biotechnol. Bioeng. 36:750-758 (1990); J. Shiloach et al, Biotechnol. Bioeng. 49:421-428 (1996)). Our observations indicate that high pyruvate dehydrogenase activity does not necessarily correlate with increased aerobic acetate production.

Under anaerobic conditions, the activity of pyruvate dehydrogenase is believed to be absent because of the low regeneration of NADH, and all the carbon from pyruvate proceeds only through pyruvate formate lyase. In the case of AFP111 and AFP111/pTrc99A-pyc, however, pyruvate is metabolized despite the inactivation of the pfl gene encoding for pyruvate-formate lyase. Moreover, aerobically induced pyruvate dehydrogenase retains activity into a subsequent anaerobic phase. A similar report of anaerobic pyruvate metabolism in E. coli by pyruvate dehydrogenase for a low in vivo ratio of NADH/NAD (M. de Graef et al., J. Bacteriol. 181:2351-2357 (1999)), demonstrates that under anaerobic conditions, pyruvate metabolism in pfl mutants is possible in the presence of CO₂ and acetate. Of course, mutants in pfl require 2-carbon intermediates for biosynthesis.

Another important enzyme is isocitrate lyase, which is necessary for carbon to flow to succinate via the glyoxylate shunt and is commonly associated with acetate metabolism. This enzyme is not active under anaerobic conditions in E. coli AFP111 or AFP111/pTrc99A-pyc (see Example XII). However, these two strains have significant isocitrate lyase activity under aerobic growth, and this activity is retained in the subsequent anaerobic production phase. For AFP111 aerobically growing at medium oxygen transfer rate, acetate accumulated to over 7 g/L, while for the other conditions acetate did not accumulate. Considering that isocitrate lyase activity was similar for the two strains and two oxygen transfer rates, it is not clear from our results why acetate would have consistently accumulated under one specific set of circumstances but not the other three.

Both AFP111 and AFP111/pTrc99A-pyc yielded the highest succinate productivities when the aerobic portion occurred at the medium oxygen transfer rate. This result suggests that the physiological role of oxygen is central to establishing succinate productivity during the anaerobic phase. The presence of oxygen is known to lead to the formation of certain harmful by-products such as peroxide, superoxide and hydroxyl radicals leading to oxidative stress. In order to overcome the oxidative stress cells can produce antioxidants such as cysteine and glutathione, which would not be required under anaerobic conditions. If such compounds are generated in the aerobic portion of a dual-phase fermentation, they may affect the subsequent anaerobic phase.

The presence of pyruvate carboxylase poses an extra burden for the cell and more energy for cell maintenance is needed in AFP111/pTrc99A-pyc than in AFP111. This additional burden would seem to account for the diminished cell growth rate. Interestingly, the presence of pyruvate carboxylase at high oxygen transfer rates prevented oxygen limitation from occurring during the entire growth phase. The presence of pyruvate carboxylase and its effect of slowing the growth rate may be the cause of decreased oxygen demand. However, at medium oxygen transfer rates, the presence of pyruvate carboxylase appears to hasten the onset of oxygen limitation. Moreover, that the RQ shifted from 0.8 to 1.2 in only one case (AFP111 with high transfer rate) suggests that the path taken by the process to oxygen limitation affects the state of the organism in the oxygen-limited stage. Additional studies with accurate measurement of specific oxygen uptake in these strains under various growth conditions limitations would seem necessary to reconcile these observations.

In summary, dual-phase fermentations permit the generation of high cell density in one phase, while generating product with high yield and productivity in a second phase. We have applied this type of fermentation to the production of succinic acid by E. coli and determined that the ideal time of transition between the growth and production phases for a desired product must be carefully selected based on physiological conditions at that moment. The final succinate yield and productivity depends greatly on the physiological state of the cells at the time of transition. Using the best transition time, fermentations achieved a final succinic acid concentration of 99.2 g/L with an overall yield of 110% and productivity of 1.3 g/Lh.

TABLE 10
Physiological milestones marking the transition between an aerobic
growth phase and an anaerobic production phase in the
fermentations of E. coli AFP111 and AFP111/pTrc99A-pyc.

| Milestone | Strain | k_{L}a (h⁻¹) | Physiological Transition Time |
|---|---|---|---|
| #1 | AFP111 | 52 | Shift to a lower, linear cell growth rate, DO about 20%, increased |
| | | | activity of fumarate reductase |
| #2 | AFP111 | 69 | DO about 40-50%, RQ remains at 0.85 |
| #3 | AFP111 | 69 | DO less than 5%, RQ has shifted to 1.25, increased activity of |
| | | | fumarate reductase and pyruvate dehydrogenase |
| #4 | AFP111/pTrc99A-pyc | 52 | Linear cell growth rate, DO has begun to decrease but is still about |
| | | | 90%. |
| #5 | AFP111/pTrc99A-pyc | 52 | Linear cell growth rate, DO about 20%, increased activity of |
| | | | fumarate reductase |
| #6 | AFP111/pTrc99A-pyc | 69 | 8.0 h |

TABLE 11
Comparison of fed-batch fermentations at the six milestones. See Table
1 for details of each milestone. Q_{P} is the volumetric succinate productivity
(g/Lh) during the anaerobic phase; q_{P} is the specific succinate productivity
(mg/gh) during the anaerobic phase; Y_{S/G} is the mass yield of succinate
based on glucose consumed during the anaerobic phase; S:A is the mass
ratio of succinate to acetate present at the end of the fermentation.
Parameters in a column followed by differing letters show statistically
significant difference at the 90% confidence level.

| Mile- | | Q_{P} | q_{P} | Y_{S/G} | S:A |
|---|---|---|---|---|---|
| stone | Strain | (g/Lh) | (mg/gh) | (g/g) | (g/g) |
| #1 | AFP111 | 1.21 ad | 72 a | 0.96 acd | 10.5 ac |
| #2 | AYP111 | 0.51 b | 35 b | 0.45 b | 6.7 ab |
| #3 | AFP111 | 0.84 c | 47 be | 0.89 a | 7.6 b |
| #4 | AFP111/pTrc99A-pyc | 1.29 a | 118 c | 1.14 c | 8.0 b |
| #5 | AFP111/pTrc99A-pyc | 1.11 d | 89 d | 1.13 c | 7.1 b |
| #6 | AFP111/pTrc99A-pyc | 0.78 c | 54 ae | 1.07 d | 10.3 c |

### EXAMPLE XIV

### Anaerobic Fermentation of S. typhimurium LT2 with and without Pyruvate Carboxylase

S. typhimurium is becoming increasingly considered as a host for the production of recombinant proteins with particular benefits of high expression levels of glycoproteins and high growth rate. S. typhimurium is a mixed acid fermenter which metabolizes glucose via the Embden-Meyerhof-Parnas pathway. Like many other prokaryotes, including E. coli, S. typhimurium grown on glucose generates the important four carbon intermediate oxaloacetate exclusively by the enzyme phosphoenolpyruvate (PEP) carboxylase. Thus, PEP carboxylase is the only enzyme serving the anaplerotic role of replenishing oxaloacetate which has been withdrawn for the synthesis of amino acids necessary for protein synthesis.

Other enzymes that exist in nature can serve such an anaplerotic role, including pyruvate carboxylase, an enzyme which converts pyruvate directly to oxaloacetate and is found in eukaryotes and some prokaryotes such as R. etli. The objective of this study was to determine how the presence of pyruvate carboxylase in S. typhimurium would affect the synthesis of oxaloacetate, cell growth and metabolism. This objective was accomplished by growing S. typhimurium under strict anaerobic conditions, in which the products of oxaloacetate are readily and unambiguously quantified. Metabolic flux analysis was used as a tool to quantify the effects.

Materials and Methods

Microorganisms and plasmids used. S. typhimurium LT2 (wild type) was used in this study. The pyc gene from R. etli was expressed using the pTrc99A-pyc plasmid, and the resulting strain is referred to as LT2-pyc. Expression of the pyc gene was induced by the presence of 1 mM isopropyl-β-D-thiogalactopyranoside (IPTG).

Media and growth conditions. Fermentations (2.0 liters in volume) were carried out in 2.5 liter BioFlo III bench top fermentors (New Brunswick Scientific, Edison, N.J.). The medium contained the following (in g/l): Luria-Bertani Miller (LB) broth, 25.0; glucose, 10.0; Na₂HPO₄.7H₂O, 3.0; KH₂PO₄, 1.5; NH₄Cl, 1.0; MgSO₄.7H₂O, 0.25; CaCl₂.2H₂O, 0.02; biotin, 0.002. Inocula for each fermentation were started from slant cultures. A 10 mL aerobic culture grown 8 hours was transferred into 100 mL of fresh medium prepared anaerobically under an atmosphere of carbon dioxide. This culture was grown 8-9 hours in sealed serum bottles at 37° C., and the 100 mL contents used to inoculate a fermenter. Each fermenter was controlled at 37° C., an impeller speed of 100 rpm, and a pH of 6.5 (using 2.0 M Na₂CO₃). Anaerobic conditions were maintained by flushing the headspace of the fermenter with oxygen-free carbon dioxide. For the strain containing the pyc gene, ampicillin was added initially to 100 mg/l IPTG was added when the optical density of the culture at 550 nm reached 0.5.

Analytical methods. Cell growth was monitored by measuring optical density (OD) at 550 nm and correlating with dry cell mass. Glucose and fermentation products were analyzed by HPLC using Coregel 64-H ion-exclusion column (Interactive chromatography, San Jose, Calif.) with 4.0 mM H₂SO₄ as mobile phase at 60° C. Cell-free extracts of S. typhimurium were prepared by centrifuging fermenter samples (8,000×g at 4° C. for 10 minutes). Cell disruption was achieved in a French pressure cell at 15,000 psi, and cell debris were removed by centrifugation (20,000×g at 4° C. for 20 minutes). Previous methods were used to determine the activities of pyruvate carboxylase, PEP carboxylase and lactate dehydrogenase. The total protein in the cell extract was also determined using the Pierce BCA reagent. Enzyme activities and protein concentrations were determined when the culture OD was approximately 1.5. An enzyme “unit” of activity is the quantity of enzyme which converts one μmol of substrate into product in one minute. Statistical comparisons were made with the Student's t-test.

Flux analysis. The methodology followed in this study to calculate intracellular fluxes has been detailed elsewhere (R. Gokarn et al., Appl Env Microbiol 66:1844-1850 (2000)).

Results

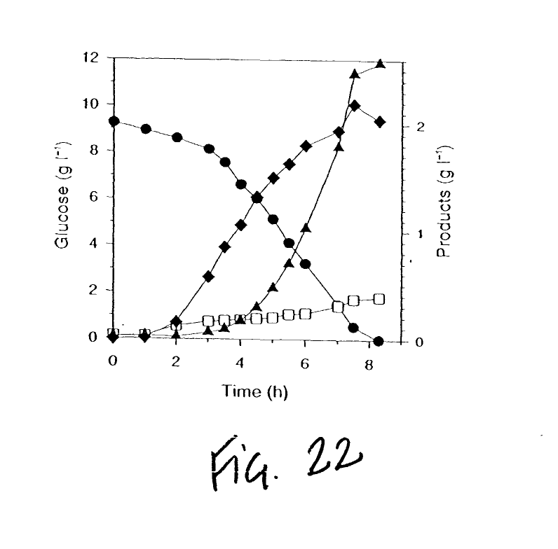

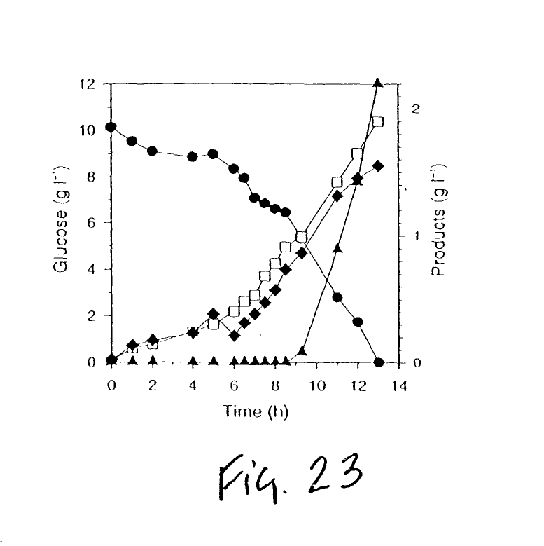

Anaerobic fermentations were performed under controlled conditions in order to assess the consequences of PYC on S. typhimurium growth, glucose consumption and product formation. Representative results for the wild type LT2 strain are shown in FIG. 22, while results for LT2-pyc are shown in FIG. 23. The LT2 strain completely consumed the initial 10 g/l glucose within 9 h, leading to a final succinate concentration of 0.40.-0.5 g/l (range of triplicate fermentations). In contrast, LT2-pyc required about 13 hours to consume the glucose, achieving a final succinate concentration of 1.6-2.6 g/l. The final concentrations of lactate and formate were also altered by the presence of active pyruvate carboxylase in S. typhimurium. For LT2 fermentations, the final lactate and formate concentrations were 2.6-3.2 g/l and 2.1-2.4 g/l, respectively. For LT2-pyc fermentations, the final lactate and formate concentrations were 0.22-2.2 g/l and 1.1-1.5 g/l, respectively. Ethanol and acetate production remained unaffected by pyruvate carboxylase activity, and these final concentrations were 1.41.7 g/l and 1.6-2.5 g/l, respectively, in both the LT2 and LT2-pyc strains. A consistent result was that the rate of lactate formation was much greater in the latter stages than in the early stages of a fermentation. This result is particularly apparent for fermentations using LT2-pyc (e.g., FIG. 23), in which lactate was generally not synthesized until 8 h, but quickly accumulated in the remaining 4-5 hours.

Fermentation results are summarized in Table 12 as average product yields. Fermentations of LT2-pyc compared to LT2 resulted in significantly greater succinate yield (P<0.01), and significantly lower yields of lactate (P<0. 10) and formate (P<0.0025). Indeed, the presence of pyruvate carboxylase in S. typhimurium led to five times the yield of succinate than in the absence of this enzyme. The yields of acetate and ethanol were not significantly affected by the presence of pyruvate carboxylase activity. These results clearly demonstrate that providing S. typhimurium LT2 with pyruvate carboxylase activity greatly altered the distribution of the anaerobic fermentation products, effectively diverting carbon from lactate and formate to succinate. The carbon recovery (i.e., carbon in products formed versus carbon in glucose consumed) was nearly 100% for the fermentations. This value was calculated considering that one mole of CO₂ was required for each mole of succinate generated, and that one mole of CO₂ was generated for each mole of acetate and ethanol (combined) generated in excess of the moles of form ate generated.

In order to understand how these yield results might be influenced by the level of the expression of the participating enzymes, we determined activities of the principal three enzymes: pyruvate carboxylase, PEP carboxylase and lactate dehydrogenase in LT2 and LT2-pyc. These enzyme activities (Table 13) were measured early in exponential growth, when the optical density of the culture was approximately 1.5 (corresponding to a dry cell mass concentration of about 0.4 g/l). Of course, LT2 did not show pyruvate carboxylase activity. Moreover, the presence of pyc resulted in approximately 50% of both PEP carboxylase activity (P<0.05) and lactate dehydrogenase activity (P<0.01) than was observed in the wild-type strain LT2. Measured enzyme activities shown in Table 13 indicate the quantity of active enzymes present, but as each of these enzyme has multiple substrate binding sites, the measurements do not indicate in vivo activities.

In order to gain insight into the changes in the partitioning of fluxes at the principal nodes in response to the metabolic perturbation, a flux analysis was performed on the fermentations of LT2 and LT2-pyc. The results indicated that carbon flux through pyruvate carboxylase was about 10 times greater than flow through PEP carboxylase during the early stages of fermentation. FIG. 23 indicates that during the early growth phase lactate is not generated. Carbon flux to ethanol and acetate was not affected by pyruvate carboxylase.

Another means to consider the impact of pyruvate carboxylase activity is to consider how carbon flux partitions at the pyruvate node during the time interval of the flux analysis. For the LT2 fermentations, 19% of the carbon flux flows to lactate and 81% flows to acetyl CoA. For the LT2-pyc fermentations, 0% of the carbon flux flows to lactate, 82% flows to acetyl CoA, and 18% to oxaloacetate. Thus, pyruvate carboxylase outcompeted lactate dehydrogenase for their mutual substrate pyruvate during this early stage of the fermentation. It is interesting to note that in LT2 fermentations, PEP carboxylase was the avenue for only 1.8% of the carbon flux from PEP, while 98% of the carbon flux led to pyruvate.

Additional information was obtained for the same time interval as the flux analysis, and Table 14 shows these results. The specific growth rate of the cells with the pyc gene was about 18% less than the wild-type cells. The specific rate of glucose consumption was about 40% less in LT2-pyc than in LT2. Calculated solely from measured data, neither of these values rely on the model of the biochemical network. A previous study showed a similar reduction in growth rate (15%) and glucose consumption (32%) comparing an E. coli ppc mutant harboring pyc to an E. coli wild-type strain (R. Gokarn et al., Appl Env Microbiol 66:1844-1850 (2000)). In another previous study using the pUC18 expression vector, however, no reduction in growth rate nor glucose consumption was observed (R. Gokarn et al. Biotechnol Lett 20:795-798 (1998)).

It may be that the high expression system used in the present study places large metabolic demands on the cell, reducing growth and glucose consumption. Since the biochemical reactions involving ATP are known in the biochemical network, the total moles of ATP generated and consumed and the rate of ATP generation and consumption can readily be calculated, and Table 14 also shows these results. The specific rate of ATP generation was 40% lower in LT2-pyc than in LT2, closely matching the difference observed in specific glucose consumption rate. This result merely confirms the direct correlation between glucose consumption and energy generation.

Although the specific rate of ATP generation was greater in LT2, the ATP yield was identical in the two strains. This result can be explained by noting that the synthesis of succinate via pyruvate carboxylase (even though this enzyme requires ATP) is energetically equivalent not only to succinate generation via PEP carboxylase, but also it is equivalent to the two other means for a cell to regenerate NAD: through the generation of ethanol or lactate. Flux analysis also permits the completion of a theoretical redox balance (R/O), a value calculated by dividing the one flux which generates NADH by the sum of all the fluxes which generate NAD (or FAD). That the redox balance is significantly lower than 1.0 for both strains suggests the existence of some unaccounted reaction, perhaps involving components of the rich media (since the carbon recoveries were about 100%).

In this example we have examined the metabolic alterations in S. typhimurium as a result of the added presence of the enzyme pyruvate carboxylase which forms oxaloacetate. The synthesis of oxaloacetate is a key step in the formation of four-carbon compounds. S. typhimurium, like E. coli, adapts to pyruvate carboxylase activity principally through the formation of lactate.

For sustained anaerobic fermentation, an organism must regenerate NAD required during glycolysis. Providing an additional means for a cell to generate NAD through succinate formation by the expression of pyruvate carboxylase would tend to reduce the intracellular pool of pyruvate as well as reduce the demand for ethanol or lactate synthesis. Pyruvate is known to be an allosteric effector of lactate dehydrogenase in E. coli. If this enzyme behaves similarly in S. typhimurium, then a slight reduction in the pyruvate pool could result in a marked reduction in lactate synthesis. Also, the enzyme activities measured for LT2 and LT2-pyc indicate that the organism adapts to the presence of pyruvate carboxylase activity by synthesizing less PEP carboxylase and lactate dehydrogenase. Thus, lactate synthesis would tend to be reduced in the pyc-containing strain both by a reduction in the level of enzyme, and a reduction of the in vivo activity of the enzyme that is present.

The reduction in PEP carboxylase activity in LT2-pyc indicates that we were conservative in our assumption of PEP remaining as a fixed node, and that an even greater fraction of the carbon flowing to succinate flows via pyruvate carboxylase than our analysis estimates. An interesting result was the significant generation of lactate in LT2-pyc fermentations during the latter stages of growth. This result may be caused by an eventual accumulation of the allosteric effector pyruvate or by a reduction in in vivo pyruvate carboxylase activity. Other operational conditions may have further reduced this level of lactate accumulation, additionally increasing succinate production.

S. typhimurium strongly prefers pyruvate carboxylase to PEP carboxylase as a means to generate oxaloacetate under anaerobic conditions, and this preference seems comparatively greater than that shown by E. coli. A similar but less-detailed study with E. coli using the pUC18 vector led to a 62% increase in succinate yield (R. Gokarn et al., Biotechnol Lett 20:795-798 (1998)), whereas over a 500% increase in succinate yield was observed in the current study using the pTrc99A vector. Moreover, the microorganisms showed nearly identical PYC activities and a similarly slight decrease in specific growth rate.

TABLE 12
Product yields and carbon recovery in
fermentation using S. typhimurium LT2.

| | | | | | | Carbon |
|---|---|---|---|---|---|---|
| | Yield (SD)^{†} | | | | | Recovery |
| Strain | Succinate | Lactate | Formate | Acetate | Ethanol | (SD) |
| LT2 | 0.04 | 0.31 | 0.23 | 0.19 | 0.17 | 0.97 |
| | (0.01) | (0.04) | (0.01) | (0.01) | (0.01) | (0.02) |
| LT2-pyc | 0.22 | 0.16 | 0.15 | 0.20 | 0.19 | 0.99 |
| | (0.07) | (0.12) | (0.02) | (0.01) | (0.02) | (0.06) |

^{†}Results are given as gram of product generated per gram of glucose consumed.

TABLE 13
Enzyme activities in cell extracts of S. typhimurium strains during
exponential growth.

| | Specific Activity (U/mg cell protein)^{†} | | |
|---|---|---|---|
| | Pyruvate | PEP | Lactate |
| Strain | carboxylase | carboxylase | dehydrogenase |
| LT2 | 0 | 0.0046 (0.0005) | 2.73 (0.16) |
| LT2-pyc | 0.069 (0.002) | 0.0020 (0.0010) | 1.47 (0.08) |

^{†}A unit (U) is the quantity of enzyme which converts one μmol of substrate into product in one minute.

TABLE 14
Metabolic data from fermentations of S. typhimurium strains during
exponential growth on glucose rich media.

| | Parameter (SD) | | | | |
|---|---|---|---|---|---|
| Strain | μ | q_{S} | q_{ATP} | Y_{ATP} | R/O |
| LT2 | 0.34 (0.05) | 16.9 (1.2) | 45.3 (2.2) | 2.68 (0.05) | 0.88 (0.00) |
| LT2- | 0.28 (0.01) | 10.1 (0.3) | 27.4 (1.3) | 2.72 (0.04) | 0.77 (0.02) |
| pyc | | | | | |

Units: μ (specific growth rate), h⁻¹; q_{S}, mmol glucose consumed/g cell · h; q^{ATP}: mmol ATP generated/g cell · h; Y_{ATP}: mole ATP formed/mole glucose consumed.

The complete disclosure of all patents, patent documents, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A metabolically engineered cell that overexpresses pyruvate carboxylase.

2. The metabolically engineered cell of claim 1 which is a bacterial cell.

3. The metabolically engineered cell of claim 1 which is a gram-negative bacterial cell.

4. The bacterial cell of claim 3 which is selected from the group consisting of a Corynebacterium glutamicum cell, an Escherichia coli cell, a Salmonella typhimurium cell, a Brevibacterium flavum cell and a Brevibacterium lactofermentum cell.

5. The bacterial cell of claim 4 which is a C. glutamicum cell.

6. The C. glutamicum cell of claim 5 having at least one of the mutations selected from the group consisting of alanine⁻, valine⁻ and acetate⁻.

7. The bacterial cell of claim 4 which is an E. coli cell.

8. The bacterial cell of claim 4 which is a S. typhimurium cell.

9. The metabolically engineered cell of claim 1 wherein a comparable wild-type of the engineered cell does not express a pyruvate carboxylase.

10. The metabolically engineered cell of claim 1 which expresses a pyruvate carboxylase derived from Rhizobium etli.

11. The metabolically engineered cell of claim 1 which expresses a pyruvate carboxylase derived from Pseudomonas fluorescens.

12. The metabolically engineered cell of claim 1 comprising a heterologous nucleic acid sequence encoding the pyruvate carboxylase.

13. The metabolically engineered cell of claim 12 wherein the heterologous nucleic acid sequence is chromosomally integrated.

14. The metabolically engineered cell of claim 1 that further overexpresses PEP carboxylase.

15. The metabolically engineered cell of claim 1 that further expresses PEP carboxykinase at a level lower than the level of PEP carboxykinase expressed in a comparable wild-type of the engineered cell.

16. The metabolically engineered cell of claim 15 that does not express a detectable level of PEP carboxykinase.

17. A metabolically engineered cell that expresses a heterologous pyruvate carboxylase.

18. The metabolically engineered cell of claim 17 which is a bacterial cell.

19. The bacterial cell of claim 18 which is selected from the group consisting of a C. glutamicum cell, an E. coli cell, an S. typhimurium cell, a B. flavum cell and a B. lactofermentum cell.

20. The bacterial cell of claim 19 which is selected from the group consisting of a C. glutamicum cell, an S. typhimurium cell and an E. coli cell.

21. The metabolically engineered cell of claim 17 that expresses a pyruvate carboxylase derived from an organism selected from the group consisting of R. etli and P. fluorescens.

22. The metabolically engineered cell of claim 17 comprising a nucleic acid sequence encoding the heterologous pyruvate carboxylase, wherein the nucleic acid sequence is chromosomally integrated.

23. The metabolically engineered cell of claim 17 wherein a comparable wild-type of the engineered cell does not express a pyruvate carboxylase.

24. The metabolically engineered cell of claim 17 that further overexpresses PEP carboxylase.

25. The metabolically engineered cell of claim 17 that further expresses PEP carboxykinase at a level lower than the level of PEP carboxykinase expressed in a comparable wild-type of the engineered cell.

26. The metabolically engineered cell of claim 25 that does not express a detectable level of PEP carboxykinase.

27. A metabolically engineered gram-negative bacterial cell that overexpresses pyruvate carboxylase.

28. A metabolically engineered cell that expresses pyruvate carboxylase, wherein a comparable wild-type of the engineered cell does not express a pyruvate carboxylase.

29. A metabolically engineered E. coli cell that expresses pyruvate carboxylase.

30. A metabolically engineered S. typhimurium cell that expresses pyruvate carboxylase.

31. A method for making a metabolically engineered cell comprising transforming a cell with a nucleic acid fragment comprising a heterologous nucleotide sequence encoding an enzyme having pyruvate carboxylase activity to yield a metabolically engineered cell that overexpresses pyruvate carboxylase.

32. The method of claim 31 comprising transforming a bacterial cell.

33. The method of claim 31 comprising transforming a gram-negative bacterial cell.

34. The method of claim 31 comprising transforming a bacterial cell selected from the group consisting of a C. glutamicum cell, an E. coli cell, an S. typhimurium cell, a B. flavum cell and a B. lactofermentum cell.

35. The method of claim 31 comprising transforming a C. glutamicum cell.

36. The method of claim 31 comprising transforming an E. coli cell.

37. The method of claim 31 comprising transforming an S. typhimurium cell.

38. The method of claim 31 comprising transforming a cell with a nucleic acid fragment comprising a nucleotide sequence selected from the group consisting of a R. etli gene encoding pyruvate carboxylase and a P. fluorescens gene encoding pyruvate carboxylase.

39. The method of claim 31 further comprising transforming the cell with a nucleic acid fragment comprising a nucleotide sequence encoding PEP carboxylase such that metabolically engineered cell overexpresses PEP carboxylase.

40. The method of claim 31 comprising transforming a metabolically engineered cell that does not express a detectable level of PEP carboxykinase.

41. A method for making a metabolically engineered cell comprising increasing the intracellular activity of an endogenous pyruvate carboxylase enzyme in a cell to yield a metabolically engineered cell that overexpresses pyruvate carboxylase.

42. The method of claim 41 wherein increasing the intracellular activity of an endogenous pyruvate carboxylase enzyme comprises transforming the cell with a nucleic acid fragment comprising a nucleotide sequence encoding the endogenous pyruvate carboxylase enzyme.

43. The method of claim 41 wherein increasing the intracellular activity of an endogenous pyruvate carboxylase enzyme comprises mutating a gene of the cell, wherein the gene encodes the endogenous pyruvate carboxylase enzyme.

44. The method of claim 41 comprising increasing the intracellular activity of an endogenous pyruvate carboxylase enzyme in a bacterial cell.

45. The method of claim 41 comprising increasing the intracellular activity of an endogenous pyruvate carboxylase enzyme in a C. glutamicum cell.

46. A method for making an oxaloacetate-derived biochemical comprising:
(a) providing a cell that produces the biochemical;
(b) transforming the cell with a nucleic acid fragment comprising a heterologous nucleotide sequence encoding an enzyme having pyruvate carboxylase activity;
(c) expressing the enzyme in the cell to cause increased production of the biochemical; and
(d) isolating the biochemical produced by the cell.

47. The method of claim 46 wherein step (a) comprises providing a bacterial cell.

48. The method of claim 46 wherein step (a) comprises providing a gram-negative bacterial cell.

49. The method of claim 46 wherein step (a) comprises providing a bacterial cell selected from the group consisting of a C. glutamicum cell, an E. coli cell, an S. typhimurium cell, a B. flavum cell and a B. lactofermentum.

50. The method of claim 46 wherein step (a) comprises providing an E. coli cell.

51. The method of claim 46 wherein step (a) comprises providing a C. glutamicum cell.

52. The method of claim 46 wherein step (a) comprises providing an S. typhimurium cell.

53. The method of claim 46 wherein step (b) comprises transforming the cell with a nucleic acid fragment comprising a heterologous nucleotide sequence selected from the group consisting of an R. etli gene encoding pyruvate carboxylase and a P. fluorescens gene encoding pyruvate carboxylase.

54. The method of claim 46 wherein step (c) comprises expressing the enzyme in the cell to cause increased production of a biochemical selected from the group consisting of an organic acid, an amino acid, a porphyrin and a pyrimidine nucleotide.

55. The method of claim 46 wherein step (c) comprises expressing the enzyme in the cell to cause increased production of a biochemical selected from the group consisting of arginine, asparagine, aspartate, glutamate, glutamine, proline, isoleucine, malate, fumarate, citrate, isocitrate, α-ketoglutarate and succinyl-CoA.

56. The method of claim 46 wherein step (c) comprises expressing the enzyme in the cell to cause increased production of lysine.

57. The method of claim 46 wherein step (c) comprises expressing the enzyme in the cell to cause increased production of succinate.

58. The method of claim 46 wherein step (c) comprises expressing the enzyme in the cell to cause increased production of threonine.

59. The method of claim 46 wherein step (c) comprises expressing the enzyme in the cell to cause increased production of methionine.

60. A method for making an oxaloacetate-derived biochemical comprising:
(a) providing a cell that produces the biochemical, wherein the cell expresses an endogenous pyruvate carboxylase;
(b) metabolically engineering the cell to yield a metabolically engineered cell that overexpresses endogenous pyruvate carboxylase;
(c) overexpressing the pyruvate carboxylase to cause increased production of the biochemical; and
(d) isolating the biochemical produced by the cell.

61. The method of claim 60 wherein step (b) comprises mutating a gene of a cell, said gene encoding the pyruvate carboxylase.

62. The method of claim 60 wherein step (b) comprises transforming the cell with a nucleic acid fragment comprising a nucleotide sequence encoding the pyruvate carboxylase.

63. The method of claim 60 wherein step (a) comprises providing a bacterial cell.

64. The method of claim 60 wherein step (a) comprises providing a C. glutamicum cell.

65. The method of claim 60 wherein step (c) comprises overexpressing the pyruvate carboxylase to cause increased production of a biochemical selected from the group consisting of an organic acid, an amino acid, a porphyrin and a pyrimidine nucleotide.

66. The method of claim 60 wherein step (c) comprises overexpressing the pyruvate carboxylase to cause increased production of a biochemical selected from the group consisting of arginine, asparagine, aspartate, glutamate, glutamine, proline, isoleucine, malate, fumarate, citrate, isocitrate, α-ketoglutarate and succinyl-CoA.

67. The method of claim 60 wherein step (c) comprises overexpressing the pyruvate carboxylase to cause increased production of lysine.

68. The method of claim 60 wherein step (c) comprises overexpressing the pyruvate carboxylase to cause increased production of succinate.

69. The method of claim 60 wherein step (c) comprises overexpressing the pyruvate carboxylase to cause increased production of threonine.

70. The method of claim 60 wherein step (c) comprises overexpressing the pyruvate carboxylase to cause increased production of methionine.

71. A method for making an oxaloacetate-derived biochemical comprising:
(a) providing a metabolically engineered cell that produces the biochemical, wherein the metabolically engineered cell overexpresses pyruvate carboxylase;
(b) anaerobically culturing the metabolically engineered cell under conditions that permit overexpression of the pyruvate carboxylase to cause increased production of the biochemical; and
(c) isolating the biochemical produced by the cell.

72. The method of claim 71 wherein step (a) comprises providing a metabolically engineered bacterial cell.

73. The method of step 71 wherein step (a) comprises providing a metabolically engineered gram-negative bacterial cell.

74. The method of claim 71 wherein step (a) comprises providing a metabolically engineered E. coli cell.

75. The method of claim 71 wherein step (a) comprises providing a metabolically engineered S. typhimurium cell.

76. The method of claim 71 wherein step (b) comprises anaerobically culturing the metabolically engineered cell to cause increased production of a biochemical selected from the group consisting of an organic acid, an amino acid, a porphyrin and a pyrimidine nucleotide.

77. The method of claim 71 wherein step (b) comprises anaerobically culturing the metabolically engineered cell to cause increased production of a biochemical selected from the group consisting of arginine, asparagine, aspartate, glutamate, glutamine, proline, isoleucine, malate, fumarate, citrate, isocitrate, α-ketoglutarate and succinyl-CoA.

78. The method of claim 71 wherein step (b) comprises anaerobically culturing the metabolically engineered cell to cause increased production of lysine.

79. The method of claim 71 wherein step (b) comprises anaerobically culturing the metabolically engineered cell to cause increased production of succinate.

80. The method of claim 71 wherein step (b) comprises anaerobically culturing the metabolically engineered cell to cause increased production of threonine.

81. The method of claim 71 wherein step (b) comprises anaerobically culturing the metabolically engineered cell to cause increased production of methionine.

82. A method for making an oxaloacetate-derived biochemical comprising:
(a) providing a metabolically engineered cell that produces the biochemical, wherein the metabolically engineered cell expresses a heterologous pyruvate carboxylase;
(b) culturing the metabolically engineered cell under conditions that permit overexpression of pyruvate carboxylase to cause increased production of the biochemical; and
(c) isolating the biochemical produced by the cell.

83. The method of claim 82 wherein step (a) comprises providing a metabolically engineered bacterial cell.

84. The method of claim 82 wherein step (a) comprises providing a metabolically engineered gram-negative bacterial cell.

85. The method of claim 82 wherein step (a) comprises providing a metabolically engineered cell selected from the group consisting of an E. coli cell, an S. typhimurium cell and a C. glutamicum cell.

86. The method of claim 82 wherein step (b) comprises culturing the metabolically engineered cell to cause increased production of a biochemical selected from the group consisting of an organic acid, an amino acid, a porphyrin and a pyrimidine nucleotide.

87. The method of claim 82 wherein step (b) comprises culturing the metabolically engineered cell to cause increased production of a biochemical selected from the group consisting of arginine, asparagine, aspartate, glutamate, glutamine, proline, isoleucine, malate, fumarate, citrate, isocitrate, α-ketoglutarate and succinyl-CoA.

88. The method of claim 82 wherein step (b) comprises culturing the metabolically engineered cell to cause increased production of lysine.

89. The method of claim 82 wherein step (b) comprises culturing the metabolically engineered cell to cause increased production of succinate.

90. The method of claim 82 wherein step (b) comprises culturing the metabolically engineered cell to cause increased production of threonine.

91. The method of claim 82 wherein step (b) comprises culturing the metabolically engineered cell to cause increased production of methionine.

92. The method of claim 82 wherein, prior to step (b), the metabolically engineered cell is cultured aerobically to increase biomass.

93. A method for making an oxaloacetate-derived biochemical comprising:
(a) providing a metabolically engineered cell that produces the biochemical, wherein the metabolically engineered cell overexpresses an endogenous pyruvate carboxylase;
(b) culturing the metabolically engineered cell under conditions that permit overexpression of the endogenous pyruvate carboxylase to cause increased production of the biochemical; and
(c) isolating the biochemical produced by the cell.

94. The method of claim 93 wherein step (a) comprises providing a metabolically engineered bacterial cell.

95. The method of claim 93 wherein step (a) comprises providing a metabolically engineered C. glutamicum cell.

96. The method of claim 93 wherein step (b) comprises culturing the metabolically engineered cell to cause increased production of a biochemical is selected from the group consisting of an organic acid, an amino acid, a porphyrin and a pyrimidine nucleotide.

97. The method of claim 93 wherein step (b) comprises culturing the metabolically engineered cell to cause increased production of a biochemical is selected from the group consisting of arginine, asparagine, aspartate, glutamate, glutamine, proline, isoleucine, malate, fumarate, citrate, isocitrate, α-ketoglutarate and succinyl-CoA.

98. The method of claim 93 wherein step (b) comprises culturing the metabolically engineered cell to cause increased production of lysine.

99. The method of claim 93 wherein step (b) comprises culturing the metabolically engineered cell to cause increased production of succinate.

100. The method of claim 93 wherein step (b) comprises culturing the metabolically engineered cell to cause increased production of threonine.

101. The method of claim 93 wherein step (b) comprises culturing the metabolically engineered cell to cause increased production of methionine.

102. A method for making succinate comprising:
(a) providing a metabolically engineered cell that produces succinate, wherein the metabolically engineered cell overexpresses pyruvate carboxylase;
(b) culturing the metabolically engineered cell under conditions that permit overexpression of the pyruvate carboxylase to cause increased production of succinate; and
(c) isolating the succinate produced by the cell.

103. The method of claim 102 wherein step (a) comprises providing a metabolically engineered bacterial cell.

104. The method of claim 102 wherein step (a) comprises providing a metabolically engineered gram-negative bacterial cell.

105. The method of claim 102 wherein step (a) comprises providing a metabolically engineered cell selected from the group consisting of an E. coli cell, an S. typhimurium cell and a C. glutamicum cell.

106. The method of claim 102 wherein step (a) comprises providing a metabolically engineered cell that overexpresses a heterologous pyruvate carboxylase.

107. The method of claim 102 further comprising metabolically engineering a cell to yield the metabolically engineered cell of step (a) that overexpresses pyruvate carboxylase

108. The method of claim 107 wherein metabolically engineering the cell comprises mutating a gene of the cell, said gene encoding the pyruvate carboxylase.

109. The method of claim 107 wherein metabolically engineering the cell comprises transforming the cell with a nucleic acid fragment comprising a nucleotide sequence encoding the pyruvate carboxylase.
